(19) United States
(12) Patent Application Publication
(10) Pub. No.: US 2025/0327135 A1
(43) Pub. Date: Oct. 23, 2025

(54) TUMOR NUCLEIC ACID IDENTIFICATION METHODS

(71) Applicant: AccuraGen Holdings Limited, Georgetown (KY)

(72) Inventors: Li WENG, San Jose, CA (US); Malek FAHAM, Burlingame, CA (US); Tobias WITTKOP, San Mateo, CA (US); Ling Fung TANG, San Francisco, CA (US)

(21) Appl. No.: 19/202,551

(22) Filed: May 8, 2025

Related U.S. Application Data
(63) Continuation of application No. PCT/US2023/078993, filed on Nov. 7, 2023.
(60) Provisional application No. 63/382,944, filed on Nov. 9, 2022.
(60) Provisional application No. 63/492,690, filed on Mar. 28, 2023.

(51) Int. Cl. C12Q 1/6886; C12Q 1/6806; C12Q 1/6855; C12Q 1/6869

(52) U.S. Cl. CPC C12Q 1/6886; C12Q 1/6806; C12Q 1/6855; C12Q 1/6869; C12Q 2600/156

(57) ABSTRACT

Provided herein are methods of detecting tumor nucleic acids in a biological sample of a subject.

### CROSS REFERENCE

This application is a continuation of PCT International Application No. PCT/US2023/078993, filed Nov. 7, 2023, which claims the benefit of U.S. Provisional Application No. 63/382,944, filed Nov. 9, 2022, and U.S. Provisional Application No. 63/492,690, filed Mar. 28, 2023, each of which is incorporated herein by reference in its entirety.

### BACKGROUND

During tumor development, nucleic acids from the tumor are often released by the tumor into the bloodstream. Apoptosis, necrosis, and active cell secretion are thought to contribute to high levels of circulating nucleic acids in the blood of some subjects with cancer.

### SUMMARY

In an aspect, provided herein are methods of detecting a tumor nucleic acid in a cell-free biological sample from a subject. In some cases, the method comprises circularizing a nucleic acid derived from the cell-free biological sample to create a circularized nucleic acid. In some cases, the method comprises amplifying the circularized nucleic acid to generate a concatemer comprising at least two copies of a sequence of the circularized nucleic acid. In some cases, the method comprises sequencing the concatemer or a derivative thereof to obtain a sequence of the concatemer, wherein the sequencing is at a depth of no greater than 18 reads. In some cases, the sequencing is a depth of no greater than 18 reads per original nucleic acid. In some cases, the sequencing is at a depth of no greater than 1 read per concatemer. In some cases, the sequencing is at a depth of no greater than 1 read per circularized nucleic acid. In some cases, the method comprises processing the sequence of the concatemer to identify at least two occurrences of a tumor specific sequence variant of the subject. In some cases, the method comprises upon identifying the at least two occurrences of the tumor specific sequence variant in the sequence of the concatemer, identifying the nucleic acid as having the at least one tumor specific sequence variant. In some cases, the method further comprises obtaining the tumor specific sequence variant from the subject. In some cases, obtaining the tumor specific sequence variant comprises sequencing nucleic acids derived from a tumor of the subject. In some cases, obtaining the tumor specific sequence variant comprises sequencing nucleic acids derived from a healthy tissue of the subject and comparing sequences of the nucleic acids derived from the tumor to sequences of the nucleic acids derived from the healthy tissue. In some cases, obtaining the tumor specific sequence variant comprises sequencing nucleic acids derived from a low or no tumor burden tissue of the subject and comparing sequences of the nucleic acids derived from the tumor to sequences derived from the low or no tumor burden tissue of the subject. In some cases, sequencing nucleic acids derived from the tumor of the subject is at a depth of greater than 20 reads. In some cases, sequencing nucleic acids derived from the tumor of the subject is at a depth of greater than 20 reads per original tumor nucleic acid molecule. In some cases, sequencing nucleic acids derived from the tumor of the subject is at a depth of greater than 20 reads per nucleotide position. In some cases, the sequencing of the concatemer is at a depth of no greater than ten reads. In some cases, the sequencing of the concatemer is at a depth of no greater than five reads. In some cases, the sequencing of the concatemer is at a depth of no greater than two reads. In some cases, the sequencing depth is measured by reads per concatemer. In some cases, the sequencing depth is measured by reads per original nucleic acid molecule. In some cases, the sequencing of the concatemer comprises at least 10 gigabases of sequence. In some cases, the sequencing of the concatemer comprises at least 10 gigabases of total sequence of the sample. In some cases, the nucleic acids derived from said tumor are subjected to selection prior to sequencing. In some cases, the nucleic acids derived from the healthy tissue is subjected to selection prior to sequencing. In some cases, selection comprises negative selection to remove non-target sequences from the nucleic acids. In some cases, selection comprises positive selection to select target sequences from the nucleic acids. In some cases, the method further comprises, prior to circularization, subjecting said nucleic acid derived from said cell-free biological sample to selection. In some cases, selection comprises negative selection to remove non-target sequences from said nucleic acids. In some cases, selection comprises positive selection to select target sequences from said nucleic acids. In some cases, circularizing comprises ligating ends of the nucleic acid or a derivative thereof to one another. In some cases, circularizing comprises coupling an adaptor to a 5′ end, a 3′ end, or a 5′ end and a 3′ end of the nucleic acid or a derivative thereof. In some cases, amplifying the circularized nucleic acid is effected by a polymerase having strand-displacement activity. In some cases, amplifying the circularized nucleic acid is effected by a polymerase having 5′ to 3′ exonuclease activity. In some cases, the amplifying is effected by at least one primer of a plurality of random primers. In some cases, the amplifying is effected by at least one primer of a plurality of primers designed for whole genome amplification. In some cases, the nucleic acid is single stranded. In some cases, the nucleic acid is double stranded. In some cases, the nucleic acid is deoxyribonucleic acid (DNA) or ribonucleic acid (RNA). In some cases, sequencing comprises (i) bringing the concatemer or a derivative thereof in contact with a plurality of nucleotides in the presence of a polymerase to incorporate one or more nucleotides of the plurality of nucleotides into a growing strands complementary to the concatemer or a derivative thereof, and (ii) detecting one or more signals indicative of incorporation of the one or more nucleotides into the growing strand. In some cases, sequencing comprises sequencing by ligation. In some cases, the tumor specific sequence variant comprises a single nucleotide variant, a fusion, an insertion, a deletion, or an epigenetic modification. In some cases, the cell-free biological sample is a bodily fluid. In some cases, the bodily fluid comprises urine, saliva, blood, serum, or plasma. In some cases, the tumor is a colorectal cancer, a pancreatic cancer, an ovarian cancer, a breast cancer, a prostate cancer, a bladder cancer, a lung cancer, a skin cancer, or a blood cancer.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1 shows an example method of tumor informed disease detection and monitoring using shallow sequencing.

FIG. 2 shows an example method of tumor informed disease detection and monitoring using shallow sequencing with concatemer-based error correction.

FIG. 3 shows a computer system that is programmed or otherwise configured to implement methods provided herein.

FIG. 4 shows an example method of genome complexity reduction.

FIG. 5A shows an example method of tumor specific mutation identification. The tumor tissue and the normal tissue (e.g., white blood cells) from the same individual are sequenced; variants are identified through comparison with human reference genome data base. Variants identified in both the normal tissue and tumor tissue are subtracted from the tumor tissue variant list; variants found in tumor tissue only are used as tumor specific variants for minimal residual disease detection in the plasma samples from the same individual.

FIG. 5B shows an example method of tumor specific mutation identification. The tumor tissue and the post-treatment plasma samples from the same individual are sequenced. The plasma sample is sequenced to certain depth (i.e., >40×, or >50×, or >60×). Variants are identified through comparison with human reference genome data base. Variants identified in the plasma with more than one molecules (or reads) support, which are also found in the tumor tissue, are subtracted from the tumor tissue variant list; variants found in tumor tissue but not found in plasma sample with more than one molecule (read) support are used as tumor specific variants for minimal residual disease detection in the plasma samples from the same individual.

FIG. 6 shows a sequencing workflow without selection. Variants are detected in the last step with single read per molecule error correction.

FIG. 7 shows a sequencing workflow with negative selection. Blocking oligonucleotides with modified 5′ and/or 3′ ends that cannot ligate nor extend are added to the ligation mix at high concentration. Blocking oligos bind to the DNA regions with complementary sequences and form double strand regions. These hybrid DNA molecules do not circularize and optionally, are removed by DNA exonuclease. Only circularized DNA will be amplified through rolling circle amplification and sequenced in the following steps. This process can be used to selectively exclude regions in the sequencing library.

FIG. 8 shows a sequencing workflow with positive selection. Primers targeting regions of interest are spiked into the WGS reaction mix including random primers. These primers bind to the target sequences in the RCA reaction and enhance the amplification for these regions of interest. Compared to the standard workflow, the regions of interest with primer spike-in are amplified more than without the primer spike-in, and as a result, these regions receive more sequencing reads in the final sequencing data.

FIG. 9A shows a workflow for whole genome sequencing with concatemer error correction.

FIG. 9B shows the error rate of WGS on healthy human cfDNA samples (N=3) measured by unfiltered reads, read1 read2 corrected reads and AccuScan.

FIG. 10A shows the limit of detection for various assay conditions.

FIG. 10B shows the false positive rate at VAF=0.

FIG. 10C shows analytical sensitivity of AccuScan using healthy sample mixtures.

FIG. 10D shows titration of cancer sample.

FIG. 11A-11B show two workflows for AccuScan MRD detection. FIG. 11A shows comparison between tumor tissue and blood cells. FIG. 11B shows comparison between tumor tissue and post-treatment plasma.

FIG. 11C shows the total number of tumor specific markers identified with and without white blood cells.

FIG. 11D shows a variant profile of tumor specific markers identified with and without white blood cells.

FIG. 11E shows comparison of VAF measured in plasma using a tumor-WBC workflow versus a tumor-plasma workflow.

FIG. 11F shows MRD calls using tumor-WBC workflow versus using tumor-plasma workflow.

FIG. 12A shows the number of tumor specific variants identified in CRC, ESCC, and melanoma.

FIG. 12B shows VAF of pre-treatment plasma samples.

FIG. 12C shows ESCC MRD Detection in One-Week PostOp Samples.

FIG. 12D shows AccuScan Detected All CRC Recurrence Before Imaging.

FIG. 12E shows Kaplan-Meier disease-free survival analysis of CRC and ESCC surgical patients. Patients who are ctDNA+ in the postOp plasma samples showed significantly shorter disease-free survival.

FIG. 13A shows AccuScan for IO monitoring.

FIG. 13B shows AccuScan for IO monitoring: ctDNA dynamic change over time.

FIGS. 14A-14B show analytical sensitivity and specificity of AccuScan. FIG. 14A shows simulation using 5000, 20000, 40000, 80000 markers and two different error rates to predict the theoretical detection rate under different sequencing coverage as a function of cTAF. The 4.2×10⁻⁷ error rate showed higher sensitivity than the 2.8×10⁻⁵ error rate under same sequencing depth. Detection rate is calculated as the fraction of test that are called MRD positive with the nominal specificity set at 99%. FIG. 14B shows simulation using 5000, 20000, 40000, 80000 markers and two different error rates to predict the theoretical specificity with the nominal specificity setting at 99%. Specificity is calculated as the fraction of tests that are called MRD negative when cTAF is 0.

FIGS. 15A-15C show ddPCR of the melanoma cancer cfDNA sample. FIG. 15A shows ddPCR of the original melanoma cancer cfDNA sample. FIG. 15B shows ddPCR of a healthy plasma sample. FIG. 15C shows ddPCR of the diluted melanoma cancer cfDNA sample in the healthy plasma background at an expected cTAF of 0.1%.

FIG. 16 shows VAF of all ctDNA positive plasma samples.

FIG. 17 shows AccuScan error rate. Overall error rate and error rate of each variant type from AccuScan WGS data on healthy human cfDNA samples (N=3) sequenced by pair end 150 read length or single end 300 read length using the 300 cycle sequencing reagents.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein the term “about” or “approximately” means within an acceptable error range for the particular value as determined by one of ordinary skill in the art, which may depend in part on how the value is measured or determined, i.e., the limitations of the measurement system. For example, “about” can mean within 1 or more than 1 standard deviation, per the practice in the art. As another example, “about” can mean a range of up to 20%, up to 10%, up to 5%, or up to 1% of a given value. With respect to biological systems or processes, the term “about” can mean within an order of magnitude, such as within 5-fold or within 2-fold of a value. Where particular values are described in the application and claims, unless otherwise stated, the term “about” means within an acceptable error range for the particular value.

As used herein, the terms “polynucleotide”, “nucleotide”, “nucleotide sequence”, “nucleic acid” and “oligonucleotide” are used interchangeably and generally refer to a polymeric form of nucleotides of any length, either deoxyribonucleotides (DNA) or ribonucleotides (RNA), or analogs thereof. Polynucleotides may have any three-dimensional structure, and may perform any function. The following are non-limiting examples of polynucleotides: cell-free nucleic acids, cell-free DNA (cfDNA), cell-free RNA (cfRNA), circulating tumor DNA (ctDNA), circulating tumor RNA (ctRNA), coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “subject,” as used herein, generally refers to a vertebrate, such as a mammal (e.g., a human). Mammals include, but are not limited to, murines, simians, humans, farm animals, sport animals, and pets (e.g., a dog or a cat). Tissues, cells, and their progeny of a biological entity obtained in vivo or cultured in vitro are also encompassed. The subject may be a patient. The subject may be symptomatic with respect to a disease (e.g., cancer). Alternatively, the subject may be asymptomatic with respect to the disease.

The term “biological sample,” as used herein, generally refers to a sample derived from or obtained from a subject, such as a mammal (e.g., a human). Biological samples may include, but are not limited to, hair, fingernails, skin, sweat, tears, ocular fluids, nasal swab or nasopharyngeal wash, sputum, throat swab, saliva, mucus, blood, serum, plasma, placental fluid, amniotic fluid, cord blood, emphatic fluids, cavity fluids, earwax, oil, glandular secretions, bile, lymph, pus, microbiota, meconium, breast milk, bone marrow, bone, CNS tissue, cerebrospinal fluid, adipose tissue, synovial fluid, stool, gastric fluid, urine, semen, vaginal secretions, stomach, small intestine, large intestine, rectum, pancreas, liver, kidney, bladder, lung, and other tissues and fluids derived from or obtained from a subject. The biological sample may be a cell-free (or cell free) biological sample.

The term “cell-free biological sample,” as used herein, generally refers to a sample derived from or obtained from a subject that is free from cells. Cell-free biological samples may include, but are not limited to, blood, serum, plasma, nasal swab or nasopharyngeal wash, saliva, urine, gastric fluid, tears, stool, mucus, sweat, earwax, oil, glandular secretion, bile, lymph, cerebrospinal fluid, tissue, semen, vaginal fluid, interstitial fluids, including interstitial fluids derived from tumor tissue, ocular fluids, spinal fluid, throat swab, breath, hair, finger nails, skin, biopsy, placental fluid, amniotic fluid, cord blood, emphatic fluids, cavity fluids, sputum, pus, microbiota, meconium, breast milk and/or other excretions.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Molecular residual disease (MRD) refers to the cancer cells persisting after curative treatment. Timely and sensitive measurement of MRD is critical for recurrence risk assessment, treatment prognosis and patient stratification. Circulating tumor DNA (ctDNA), which is released by cancer cells and has a short half-life (<2 hours), has emerged as a promising real-time biomarker for MRD detection and monitoring. Studies have shown that levels of cancer-specific somatic mutations in ctDNA correlate with tumor stage, burden, and response to therapy across tumor types. Compared to other blood-based cancer biomarkers, such as circulating tumor cells and cancer antigens, ctDNA provides a more sensitive and specific measure of MRD.

There are currently two main strategies for ctDNA-based MRD detection: 1) the tumor-naïve approach, which tests MRD samples for changes known to be enriched in tumors, such as common somatic mutations and methylation changes and 2) the tumor-informed approach, which requires a tumor sample to identify patient specific variants and then tests MRD samples for those variants.

The tumor-naïve approach is logistically simple, without the need to acquire and sequence a tumor sample and uses a universal panel to test the plasma samples for the presence of a cancer signal. While these tests offer operational convenience, they tend to have moderate limit of detection (LOD). With a methylation-based cancer detection test, a 50% sensitivity to a 3.1×10⁻⁴ circulating tumor allele fraction (cTAF) has been claimed. It has also been shown that 55.6% detection of CRC recurrence using plasma collected at landmark time point (4 week after surgery) with a panel combining methylation and mutation signals.

The tumor informed approach incorporates patient-specific somatic mutation information from the tumor tissue into the MRD analysis, which can lead to ultra-low detection limit. Factors that impact its sensitivity include the accuracy of somatic mutation calls from the tissue and plasma samples, and the total number of cfDNA molecules interrogated, which is the product of the number of somatic variants tracked and the unique molecular depth obtained through sequencing.

Tumor informed approaches can either use a bespoke or off-the-shelf MRD test. A bespoke MRD assay is designed after tumor results are available and follows a limited number of variants through ultra-deep sequencing. The sequencing of a bespoke panel can be exhaustive; hence the unique molecular depth is mostly limited by the amount of available input material. For example, Signatera, a tumor-informed NGS-based multiplex PCR assay that tracks 16 personalized markers achieved 81.3%-96.1% analytical sensitivity at limit of detection (LOD) of 10⁻⁴ when up to 66 ng of DNA is used. Tumor-informed personalized MRD assays targeting large numbers of markers and boasting error correction using UMI or duplex sequencing have shown LOD below 10⁻⁴. Phase-Seq uses multiple somatic mutations in individual DNA fragments for detecting ctDNA, which lowered the background noise to less than 10⁻⁶ and claimed limit of detection down to the PPM level given enough phased variants. While the tumor-informed bespoke MRD approach may achieve very high sensitivity, the requirement of a personalized design substantially increases turnaround time (TAT) and creates considerable logistical challenges.

The tumor informed off-the-shelf method uses the same assay for both tumor and plasma in all patients. Without the need of patient specific reagents, it shares the low TAT of a tumor-naïve approach and offers a much simpler logistics than the be-spoke method. The challenge is generating an off-the-shelf assay that covers enough of the genome at a low enough error rate. Pre-designed MRD panels targeting cancer-related genes typically use UMI with deep sequencing to achieve high accuracy in variant call, but the number of markers these panel track for each patient is sparse. For example, a 130 kb panel covering 139 critical lung cancer-related genes only captures a median of 2 mutations per patient (range: 1-8 mutations.

Whole genome sequencing (WGS) assays have recently emerged as an innovative approach for cancer screening and MRD detection. Tumor-informed WGS MRD assays use genome breadth to supplement sequencing depth for sensitivity, overcoming the limitation of input sample amount. UMI-based error correction, which relies on having multiple reads per input molecule, would be cost prohibitive on a WGS scale. Some have used a read-centric SVM model to reduce WGS somatic single-nucleotide variants (SNV) error rate to 4.96×10⁻⁵. By capitalizing on the cumulative signal of thousands of somatic mutations observed in the tumor genome, they reported a 95% analytical sensitivity at tumor fraction of 10⁻⁴. Other whole genome technologies using duplex sequencing have demonstrated ultra-low error rate at <10⁴ level, however, these methods suffer from low conversion rates, making a low LOD difficult to achieve. There is a need for an efficient and cost-effective genome-wide error correction method to enable WGS for MRD detection with low LOD

DNA concatemers generated via rolling circle amplification (RCA) physically link DNA copies, allowing error correction at single read level. The combination of RCA with repeat confirmation eliminates both PCR and sequencing errors. Compared to UMI methods, concatemer sequencing has shown higher efficiency in error correction when applied to genomic DNA. Recently, concatemer sequencing has been adapted for liquid biopsy to demonstrate feasibility of applying the technology to therapy selection and cancer screen. Provided herein is a WGS solution for ctDNA detection that utilizes concatemer sequencing for genome wide single-read error suppression, enabling fast and sensitive MRD detection and monitoring in cancer patient plasma samples.

Provided herein, in an aspect, are methods of detecting a tumor nucleic acid in a biological sample from a subject. In some cases, the method comprises detecting the tumor nucleic acid in a cell-free biological sample from a subject. In some cases, the method comprises circularizing a nucleic acid derived from the biological sample, such as the cell-free biological sample, to create a circularized nucleic acid. Next, the method can comprise amplifying the circularized nucleic acid to generate a concatemer comprising at least two copies of a sequence of the circularized nucleic acid. Then, the concatemer or a derivative thereof can be sequenced to obtain a sequence of the concatemer. In some cases, the sequencing is at a depth of no greater than 18 reads. Next, the sequence of the concatemer is processed to identify at least two occurrences of a tumor specific sequence variant of the subject. Upon identifying the at least two occurrences of the tumor specific sequence variant in the sequence of the concatemer, the method can comprise identifying the nucleic acid as having the at least one tumor specific sequence variant. The method can further comprise obtaining the tumor specific sequence variant from the subject, for example by sequencing nucleic acids derived from a tumor of the subject. In some cases, the method further comprises sequencing nucleic acids derived from a healthy tissue of the subject and comparing sequence from the nucleic acids derived from the tumor to sequence from the nucleic acids derived from the healthy tissue of the subject. In some cases, the sequencing of nucleic acids derived from the tumor is done at a suitable depth measured in reads per molecule or reads, used interchangeably herein. In some cases, the sequencing of nucleic acids derived from the tumor of the subject is at a depth of greater than 20 reads. In some cases, the sequencing of nucleic acids derived from the tumor of the subject is at a depth of greater than 25 reads. In some cases, the sequencing of nucleic acids derived from the tumor of the subject is at a depth of greater than 30 reads. In some cases, the sequencing of nucleic acids derived from the tumor of the subject is at a depth of greater than 35 reads. In some cases, the sequencing of nucleic acids derived from the tumor of the subject is at a depth of greater than 40 reads.

In another aspect of methods of detecting a tumor nucleic acid in a biological sample herein, sequencing of the concatemer is done at a suitable depth measured in reads per molecule or reads, used interchangeably herein. In some cases, sequencing of the concatemer is at a depth of no greater than 18 reads. In some cases, sequencing of the concatemer is at a depth of no greater than 15 reads. In some cases, sequencing of the concatemer is at a depth of no greater than 12 reads. In some cases, sequencing of the concatemer is at a depth of no greater than 10 reads. In some cases, sequencing of the concatemer is at a depth of no greater than nine reads. In some cases, sequencing of the concatemer is at a depth of no greater than eight reads. In some cases, sequencing of the concatemer is at a depth of no greater than seven reads. In some cases, sequencing of the concatemer is at a depth of no greater than six reads. In some cases, sequencing of the concatemer is at a depth of no greater than five reads. In some cases, sequencing of the concatemer is at a depth of no greater than four reads. In some cases, sequencing of the concatemer is at a depth of no greater than three reads. In some cases, sequencing of the concatemer is at a depth of no greater than two reads. In some cases, sequencing of the concatemer is at a depth of no greater than one read. In some cases, sequencing of the concatemer is whole genome sequencing. In some cases, sequencing of the concatemer comprises at least 10 gigabases of sequence.

In another aspect of detecting a tumor nucleic acid in a biological sample herein, the nucleic acids derived from the tumor are subjected to selection prior to sequencing. In some cases, the nucleic acids derived from the healthy tissue are subjected to selection prior to sequencing. In some cases, prior to circularizing nucleic acids, the nucleic acid derived from the cell-free biological sample is subjected to selection. In some cases, selection comprises negative selection to remove non-target sequences from said nucleic acids. In some cases, negative selection comprises contacting the nucleic acids with a blocker that binds to the non-target sequences and amplifying, ligating, or capturing nucleic acids that are not bound to the blocker. In some cases, the blocker comprises an oligonucleotide. In some cases, negative selection comprises contacting the nucleic acids with a nuclease that specifically cleaves the non-target sequences. In some cases, the nuclease is a clustered regularly interspaced short palindromic repeats (CRISPR) nuclease. In some cases, selection comprises positive selection to select target sequences from said nucleic acids. In some cases, positive selection comprises hybrid capture. In some cases, positive selection comprises amplification. In some cases, amplification comprises polymerase chain reaction (PCR).

In a further aspect of methods of detecting a tumor nucleic acid in a biological sample herein, in some cases, circularizing the nucleic acid derived from the biological sample comprises ligating ends of the nucleic acid or a derivative thereof to one another. In some cases, circularizing the nucleic acid derived from the biological sample comprises coupling an adaptor to a 5′ end, a 3′ end, or a 5′ end and a 3′ end of the nucleic acid or a derivative thereof.

In another aspect of methods of detecting a tumor nucleic acid in a biological sample herein, in some cases, amplification of the circularized nucleic acid to generate a concatemer a is effected by a polymerase having strand-displacement activity. In some cases, amplification of the circularized nucleic acid to generate a concatemer is effected by a polymerase having 5′ to 3′ exonuclease activity. In some cases, amplifying is effected by at least one primer of a plurality of random primers. In some cases, amplifying is effected by at least one primer of a plurality of primers designed for whole genome amplification.

In another aspect of methods of detecting a tumor nucleic acid in a biological sample herein, in some cases, the nucleic acid in the biological sample is single stranded. In some cases, the nucleic acid is double stranded. In some cases, the nucleic acid in the biological sample is a mixture of single stranded and double stranded nucleic acids. In some cases, the nucleic acid is made single stranded prior to circularization. In some cases, the nucleic acid is deoxyribonucleic acid (DNA), ribonucleic acid (RNA), or a combination of DNA and RNA.

In another aspect of methods of detecting a tumor nucleic acid in a biological sample herein, in some cases sequencing the concatemer comprises (bringing the concatemer or a derivative thereof in contact with a plurality of nucleotides in the presence of a polymerase to incorporate one or more nucleotides of the plurality of nucleotides into a growing strands complementary to the concatemer or a derivative thereof, and detecting one or more signals indicative of incorporation of the one or more nucleotides into the growing strand. Alternatively, or in combination, sequencing the concatemer comprises sequencing by ligation. Sequencing of the concatemer can comprise any suitable method provided herein.

In another aspect of methods of detecting a tumor nucleic acid in a biological sample provided herein, in some cases, the tumor specific sequence variant comprises a single nucleotide variant, a fusion, an insertion, a deletion, an epigenetic modification, or any combination thereof.

In another aspect of methods of detecting a tumor nucleic acid in a biological sample provided herein, in some cases, the biological sample is a cell-free biological sample. In some cases, the cell-free biological sample is a bodily fluid. In some cases, the bodily fluid comprises urine, saliva, blood, serum, or plasma. In some cases, the biological sample, cell-free biological sample, or bodily fluid is any suitable sample provided herein.

In another aspect of methods of detecting a tumor nucleic acid in a biological sample provided herein, in some cases, the tumor is a colorectal cancer, a pancreatic cancer, an ovarian cancer, a breast cancer, a prostate cancer, a bladder cancer, a lung cancer, a skin cancer, or a blood cancer. In some cases, the tumor is any cancer suitable for detection provided herein.

### Methods of Library Preparation and Amplification

Methods of detecting a tumor nucleic acid provided herein comprise, in certain cases, amplification of polynucleotides present in a sample from a subject. Methods of amplification used herein often comprise rolling-circle amplification. Alternatively or in combination, methods of amplification used herein comprise PCR. In some cases, methods of amplification herein comprise linear amplification. Often amplification is not targeted to one gene or set of genes and the entire nucleic acid sample is amplified. In some cases, the method comprises (a) circularizing individual polynucleotides of the plurality to form a plurality of circular polynucleotides, each of which having a junction between the 5′ end and the 3′ end; and (b) amplifying the circular polynucleotides of (a) to produce amplified polynucleotides. In additional cases, methods of amplification comprise (c) shearing the amplified polynucleotides to produce sheared polynucleotides, each sheared polynucleotide comprising one or more shear points at a 5′ end and/or 3′ end. In some cases, the method does not comprise enriching for a target sequence.

In general, joining ends of a polynucleotide to one-another to form a circular polynucleotide (either directly, or with one or more intermediate adapter oligonucleotides) produces a junction having a junction sequence. Where the 5′ end and 3′ end of a polynucleotide are joined via an adapter polynucleotide, the term “junction” can refer to a junction between the polynucleotide and the adapter (e.g. one of the 5′ end junction or the 3′ end junction), or to the junction between the 5′ end and the 3′ end of the polynucleotide as formed by and including the adapter polynucleotide. Where the 5′ end and the 3′ end of a polynucleotide are joined without an intervening adapter (e.g. the 5′ end and 3′ end of a single-stranded DNA), the term “junction” refers to the point at which these two ends are joined. A junction may be identified by the sequence of nucleotides comprising the junction (also referred to as the “junction sequence”).

Samples herein comprise polynucleotides having a mixture of ends formed by natural degradation processes (such as cell lysis, cell death, and other processes by which polynucleotides such as DNA and RNA are released from a cell to its surrounding environment in which it may be further degraded, e.g., cell-free polynucleotides, e.g., cell-free DNA and cell-free RNA), fragmentation that is a byproduct of sample processing (such as fixing, staining, and/or storage procedures), and fragmentation by methods that cleave DNA without restriction to specific target sequences (e.g. mechanical fragmentation, such as by sonication; non-sequence specific nuclease treatment, such as DNase I, fragmentase). Where samples comprise polynucleotides having a mixture of ends, the likelihood of two polynucleotides having the same 5′ end or 3′ end is low, and the likelihood that two polynucleotides will independently have both the same 5′ end and 3′ end is lower. Accordingly, in some embodiments, junctions may be used to distinguish different polynucleotides, even where the two polynucleotides comprise a portion having the same target sequence. Where polynucleotide ends are joined without an intervening adapter, a junction sequence may be identified by alignment to a reference sequence. For example, where the order of two component sequences appears to be reversed with respect to the reference sequence, the point at which the reversal appears to occur may be an indication of a junction at that point. Where polynucleotide ends are joined via one or more adapter sequences, a junction may be identified by proximity to the known adapter sequence, or by alignment as above if a sequencing read is of sufficient length to obtain sequence from both the 5′ and 3′ ends of the circularized polynucleotide. In some embodiments, the formation of a particular junction is a sufficiently rare event such that it is unique among the circularized polynucleotides of a sample.

In some embodiments, circularizing individual polynucleotides in (a) is effected by subjected the plurality of polynucleotides to a ligation reaction. The ligation reaction may comprise a ligase enzyme. In some cases, the ligase enzyme is a single strand DNA or RNA ligase. In some cases, the ligase enzyme is a double strand DNA ligase. In some embodiments, the ligase enzyme is degraded prior to amplifying in (b). Degradation of ligase prior to amplifying in (b) can increase the recovery rate of amplifiable polynucleotides. In some embodiments, the plurality of circularized polynucleotides is not purified or isolated prior to (b). In some embodiments, uncircularized, linear polynucleotides are degraded prior to amplifying. In some cases, the plurality of polynucleotides is denatured to create single stranded polynucleotides prior to circularization; in some cases, the plurality of the polynucleotides is not denatured prior to circularization.

In some cases, circularizing in (a) comprises the step of joining and adapter polynucleotide to the 5′ end, the 3′ end, or both the 5′ end and the 3′ end of a polynucleotide in the plurality of polynucleotides. As previously described, where the 5′ end and/or 3′ end of a polynucleotide are joined via an adapter polynucleotide, the term “junction” can refer to the junction between the polynucleotide and the adapter (e.g., one of the 5′ end junction or the 3′ end junction), or to the junction between the 5′ end and the 3′ end of the polynucleotide as formed by and including the adapter polynucleotide.

In some cases, polynucleotides are subjected to a selection step. In some cases, polynucleotides having a sequence of interest are subjected to a positive selection step to enrich for the polynucleotides having the sequence of interest. Alternatively, polynucleotides having an unwanted sequence are subjected to a negative selection step to remove the polynucleotides having an unwanted sequence. In some cases, the negative selection comprises denaturing the polynucleotides to create single stranded polynucleotides, annealing one or more blocking oligonucleotides to the polynucleotides to create double stranded polynucleotides having the unwanted sequences and single stranded polynucleotides, and circularizing the single stranded polynucleotides. In some cases, the blocking oligonucleotides have a modified 5′ end and/or a modified 3′ end that does not allow ligation. In some cases, the blocking oligonucleotides have a modified 5′ end and/or a modified 3′ end that does not allow extension. In some cases, the linear double stranded polynucleotides are removed using an exonuclease. The circularized polynucleotides can be used in subsequent steps of rolling circle amplification and sequencing.

In one aspect, provided herein is a method of identifying a sequence variant in a plurality of polynucleotides comprising denaturing the plurality of polynucleotides, annealing one or more blocking oligonucleotides to polynucleotides having an unwanted sequence, and circularizing the resulting single stranded polynucleotides. In some cases, the remaining linear polynucleotides annealed to the blocking oligonucleotides are degraded, for example using a nuclease, such as a DNA exonuclease. Next, the circularized polynucleotides can be amplified by rolling circle amplification resulting in concatemers containing more than one copy of the original polynucleotide. In some cases, rolling circle amplification is effected with random primers. In some cases, rolling circle amplification is effected with target specific primers. Next the concatemers are subjected to sequencing to obtain sequencing reads. These sequencing reads are used to identify variants. In some cases, the variant is identified when it is present on more than one copy of the polynucleotide in the concatemer. In some cases, the variant is identified when it is present on two different concatemers.

The circularized polynucleotides are amplified, in some cases, for example, after degradation of the ligase enzyme, to yield amplified polynucleotides. Amplifying the circular polynucleotides in (b) can be effected by a polymerase. In some cases, the polymerase is a polymerase having strand-displacement activity. In some cases, the polymerase is a Phi29 DNA polymerase. Alternatively, the polymerase is a polymerase that does not have strand-displacement activity. In some cases, the polymerase is a T4 DNA polymerase or a T7 DNA polymerase. Alternately or in combination, the polymerase is a Taq polymerase, or polymerase in the Taq polymerase family. In some cases, amplification comprises rolling circle amplification (RCA). The amplified polynucleotides resulting from RCA can comprise linear concatemers, or polynucleotides comprising more than one copy of a target sequence (e.g., subunit sequence) from a template polynucleotide. In some embodiments, amplifying comprises subjecting the circular polynucleotides to an amplification reaction mixture comprising random primers. In some cases, amplifying comprises subjecting the circular polynucleotides to an amplification reaction mixture comprising one or more primers, each of which specifically hybridizes to a different target sequence via sequence complementarity. In some cases, amplifying comprises subjecting the circular polynucleotides to an amplification reaction mixture comprising inverse primers.

The amplified polynucleotides are sheared, in some cases, to produce sheared polynucleotides that are shorter in length relative to the unsheared polynucleotides. Two or more sheared polynucleotides originating from the same linear concatemer may have the same junction sequence but can have different 5′ and/or 3′ ends (e.g., shear ends).

Cell-free polynucleotides from a sample may be any of a variety of polynucleotides, including but not limited to, DNA, RNA, ribosomal RNA (rRNA), transfer RNA (tRNA), micro RNA (miRNA), messenger RNA (mRNA), small interfering RNA (siRNA), fragments of any of these, or combinations of any two or more of these. In some embodiments, samples comprise DNA. In some embodiments, samples comprise cell-free genomic DNA. In some embodiments, the samples comprise DNA generated by amplification, such as by primer extension reactions using any suitable combination of primers and a DNA polymerase, including but not limited to polymerase chain reaction (PCR), reverse transcription, and combinations thereof. Where the template for the primer extension reaction is RNA, the product of reverse transcription is referred to as complementary DNA (cDNA). Primers useful in primer extension reactions can comprise sequences specific to one or more targets, random sequences, partially random sequences, and combinations thereof. In some cases, primers comprise a mixture of random sequences and sequences specific to one or more targets. In general, sample polynucleotides comprise any polynucleotide present in a sample, which may or may not include target polynucleotides. The polynucleotides may be single-stranded, double-stranded, or a combination of these. In some embodiments, polynucleotides subjected to a method of the disclosure are single-stranded polynucleotides, which may or may not be in the presence of double-stranded polynucleotides. In some embodiments, the polynucleotides are single-stranded DNA. Single-stranded DNA (ssDNA) may be ssDNA that is isolated in a single-stranded form, or DNA that is isolated in double-stranded form and subsequently made single-stranded for the purpose of one or more steps in a method of the disclosure.

In one aspect, provided herein is a method of identifying a sequence variant in a plurality of polynucleotides comprising denaturing the polynucleotides, circularizing the resulting linear polynucleotides, and amplifying the resulting circular polynucleotides, the amplification step is used to enrich for sequences of interest, for example by adding one or more primers that bind to sequences of interest to the amplification reaction comprising random primers. The random primers and the primers binding the sequences of interest are used to amplify the circular polynucleotides by rolling circle amplification to create concatemers. Next the concatemers are subjected to sequencing to obtain sequencing reads. These sequencing reads are used to identify variants. In some cases, the variant is identified when it is present on more than one copy of the polynucleotide in the concatemer. In some cases, the variant is identified when it is present on two different concatemers.

In some embodiments, polynucleotides are subjected to subsequent steps (e.g. circularization and amplification) without an extraction step, and/or without a purification step. For example, a fluid sample may be treated to remove cells without an extraction step to produce a purified liquid sample and a cell sample, followed by isolation of DNA from the purified fluid sample. A variety of procedures for isolation of polynucleotides are available, such as by precipitation or non-specific binding to a substrate followed by washing the substrate to release bound polynucleotides. Where polynucleotides are isolated from a sample without a cellular extraction step, polynucleotides will largely be extracellular or “cell-free” polynucleotides, such as cell-free DNA and cell-free RNA, which may correspond to dead or damaged cells. The identity of such cells may be used to characterize the cells or population of cells from which they are derived, such as tumor cells (e.g. in cancer detection), fetal cells (e.g. in prenatal diagnostic), cells from transplanted tissue (e.g. in early detection of transplant failure), or members of a microbial community.

If a sample is treated to extract polynucleotides, such as from cells in a sample, a variety of extraction methods are available. For example, nucleic acids can be purified by organic extraction with phenol, phenol/chloroform/isoamyl alcohol, or similar formulations, including TRIzol and TriReagent. Other non-limiting examples of extraction techniques include: (1) organic extraction followed by ethanol precipitation, e.g., using a phenol/chloroform organic reagent (Ausubel et al., 1993, which is entirely incorporated herein by reference), with or without the use of an automated nucleic acid extractor, e.g., the Model 341 DNA Extractor available from Applied Biosystems (Foster City, Calif); (2) stationary phase adsorption methods (U.S. Pat. No. 5,234,809; Walsh et al., 1991, each of which is entirely incorporated herein by reference); and (3) salt-induced nucleic acid precipitation methods (Miller et al., (1988) which is entirely incorporated herein by reference), such precipitation methods being typically referred to as “salting-out” methods. Another example of nucleic acid isolation and/or purification includes the use of magnetic particles to which nucleic acids can specifically or non-specifically bind, followed by isolation of the beads using a magnet, and washing and eluting the nucleic acids from the beads (see e.g. U.S. Pat. No. 5,705,628, which is entirely incorporated herein by reference). In some embodiments, the above isolation methods may be preceded by an enzyme digestion step to help eliminate unwanted protein from the sample, e.g., digestion with proteinase K, or other like proteases. See, e.g., U.S. Pat. No. 7,001,724, which is entirely incorporated herein by reference. If desired, Rnase inhibitors may be added to the lysis buffer. For certain cell or sample types, it may be desirable to add a protein denaturation/digestion step to the protocol. Purification methods may be directed to isolate DNA, RNA, or both. When both DNA and RNA are isolated together during or subsequent to an extraction procedure, further steps may be employed to purify one or both separately from the other. Sub-fractions of extracted nucleic acids can also be generated, for example, purification by size, sequence, or other physical or chemical characteristic. In addition to an initial nucleic acid isolation step, purification of nucleic acids can be performed after any step in the disclosed methods, such as to remove excess or unwanted reagents, reactants, or products. A variety of methods for determining the amount and/or purity of nucleic acids in a sample are available, such as by absorbance (e.g. absorbance of light at 260 nm, 280 nm, and a ratio of these) and detection of a label (e.g. fluorescent dyes and intercalating agents, such as SYBR green, SYBR blue, DAPI, propidium iodine, Hoechst stain, SYBR gold, ethidium bromide).

In some cases, methods herein comprise preparation of a DNA library from polynucleotides. For example, methods herein comprise preparation of a single stranded DNA library. Any suitable method of preparing a single stranded DNA library may be used in methods herein. For example, the method of preparing a single stranded DNA library comprises denaturing the DNA sample to create a plurality of ssDNA; ligating an adapter to the 3′ end of the ssDNA molecules or extending the 3′ end of the ssDNA molecules through a non-template synthesis; synthesizing a second strand using a primer complementary to the adapter or the 3′ extended sequence; ligating a double stranded adapter to the extension products; amplifying the second strand using primers targeting the first and second adapters (for example, using PCR); and sequencing the library on a sequencer. An additional method of single stranded library preparation comprises denaturing the DNA sample to create a plurality of ssDNA; ligating an adapter to the 3′ end of the ssDNA molecules; synthesizing the second strand by using a primer complementary to the adapter; ligating a double stranded adapter to the extension products; amplifying the second strand (for example, by PCR) using primers targeting the first and second adapters; optionally enriching for the regions of interest using hybridization with capture probes; amplifying (for example, by PCR) the captured products; and sequencing the library on a sequencer.

Further examples of single stranded library preparation include a method comprising the steps of treating the DNA with a heat labile phosphatase to remove residual phosphate groups from the 5′ and 3′ ends of the DNA strands; removal of deoxyuracils derived from cytosine deamination from the DNA strands; ligation of a 5′-phosphorylated adapter oligonucleotide having about 10 nucleotides and a long 3′ biotinylated spacer arm to the 3′ ends of the DNA strands; immobilization of adapter-ligated molecules on streptavidin beads; copying the template strand using a 5′-tailed primer complementary to the adapter using Bst polymerase; washing away excess primers; removal of 3′ overhangs using T4 DNA polymerase; joining a second adapter to the newly synthesized strands using blunt-end ligation; washing away excess adapter; releasing library molecules by heat denaturation; adding full-length adapter sequences including bar codes through amplification using tailed primers; and sequencing the library, as described in Gansauge et al. 2013. Nature Protocols. 8(4) 737-748, which is entirely incorporated herein by reference.

In additional embodiments, methods herein comprise preparation of a double stranded DNA library. Any suitable method of preparing a double stranded DNA library may be used in methods herein. For example, the method of preparing a double stranded DNA library comprises ligating sequencing adapters to the 5′ and 3′ ends of a plurality of DNA fragments and sequencing the library on a sequencer. An additional method of double stranded DNA library preparation comprises ligating adapters to the 5′ and 3′ ends of a plurality of DNA fragments; attaching the full adapter sequences to the ligated fragments through PCR using primers that are complementary to the ligated adapters; and sequencing the library on a sequencer. A further method comprises ligating adapters to the 5′ and 3′ ends of a plurality of DNA fragments; amplifying the ligated product through PCR that are complementary to the ligated adapters; optionally enriching for the regions of interest through hybridization with capture probes; PCR amplifying the captured products; and sequencing the library on a sequencer. An additional method of double stranded library preparation comprises ligating adapters to the 5′ and 3′ ends of a plurality of DNA fragments; amplifying the ligated product through PCR using primers that are complementary to the ligated adapters; circularizing the double stranded PCR products or denature and circularize the single stranded PCR products; optionally enriching for the regions of interest by PCR using primers targeting specific genes; and sequencing the library on a sequencer.

Further examples of double stranded library preparation include the Safe-Sequencing System described in Kinde et al. (Kinde et al. 2011. Proc. Natl. Acad. Sci., USA, 108(23) 9530-9535, which is entirely incorporated herein by reference) which comprises assignment of a unique identifier (UID) to each template molecule; amplification of each uniquely tagged template molecule to create UID families; and redundant sequencing of the amplification products. An additional example comprises the circulating single-molecule amplification and resequencing technology (cSMART) described in Lv et al. (Lv et al. 2015. Clin. Chem., 61(1) 172-181, which is entirely incorporated herein by reference) which tags single molecules with unique barcodes, circularizes, targets alleles for replication by inverse PCR, then sequencing the prepared library and counts the alleles present.

In additional library preparation methods, cfDNA fragments having certain features are selected using an antibody. In some cases, cfDNA fragments that are methylated or hypermethylated are selected using an antibody. Selected cfDNA fragments are then used in any library preparation method described herein, including circularization, single stranded DNA library preparation, and double stranded DNA library preparation. Sequencing such isolated cfDNA fragments provides information as to the features present in the cfDNA, including modifications such as methylation or hypermethylation.

According to some embodiments, polynucleotides among the plurality of polynucleotides from a sample are circularized. Circularization can include joining the 5′ end of a polynucleotide to the 3′ end of the same polynucleotide, to the 3′ end of another polynucleotide in the sample, or to the 3′ end of a polynucleotide from a different source (e.g. an artificial polynucleotide, such as an oligonucleotide adapter). In some embodiments, the 5′ end of a polynucleotide is joined to the 3′ end of the same polynucleotide (also referred to as “self-joining”). In some embodiment, conditions of the circularization reaction are selected to favor self-joining of polynucleotides within a particular range of lengths, so as to produce a population of circularized polynucleotides of a particular average length. For example, circularization reaction conditions may be selected to favor self-joining of polynucleotides shorter than about 5000, 2500, 1000, 750, 500, 400, 300, 200, 150, 100, 50, or fewer nucleotides in length. In some embodiments, fragments having lengths between 50-5000 nucleotides, 100-2500 nucleotides, or 150-500 nucleotides are favored, such that the average length of circularized polynucleotides falls within the respective range. In some embodiments, 80% or more of the circularized fragments are between 50-500 nucleotides in length, such as between 50-200 nucleotides in length. Reaction conditions that may be optimized include the length of time allotted for a joining reaction, the concentration of various reagents, and the concentration of polynucleotides to be joined. In some embodiments, a circularization reaction preserves the distribution of fragment lengths present in a sample prior to circularization. For example, one or more of the mean, median, mode, and standard deviation of fragment lengths in a sample before circularization and of circularized polynucleotides are within 75%, 80%, 85%, 90%, 95%, or more of one another.

In some cases, rather than preferentially forming self-joining circularization products, one or more adapter oligonucleotides are used, such that the 5′ end and 3′ end of a polynucleotide in the sample are joined by way of one or more intervening adapter oligonucleotides to form a circular polynucleotide. For example, the 5′ end of a polynucleotide can be joined to the 3′ end of an adapter, and the 5′ end of the same adapter can be joined to the 3′ end of the same polynucleotide. An adapter oligonucleotide includes any oligonucleotide having a sequence, at least a portion of which is known, that can be joined to a sample polynucleotide. Adapter oligonucleotides can comprise DNA, RNA, nucleotide analogues, non-canonical nucleotides, labeled nucleotides, modified nucleotides, or combinations thereof. Adapter oligonucleotides can be single-stranded, double-stranded, or partial duplex. In general, a partial-duplex adapter comprises one or more single-stranded regions and one or more double-stranded regions. Double-stranded adapters can comprise two separate oligonucleotides hybridized to one another (also referred to as an “oligonucleotide duplex”), and hybridization may leave one or more blunt ends, one or more 3′ overhangs, one or more 5′ overhangs, one or more bulges resulting from mismatched and/or unpaired nucleotides, or any combination of these. When two hybridized regions of an adapter are separated from one another by a non-hybridized region, a “bubble” structure results. Adapters of different kinds can be used in combination, such as adapters of different sequences. Different adapters can be joined to sample polynucleotides in sequential reactions or simultaneously. In some embodiments, identical adapters are added to both ends of a target polynucleotide. For example, first and second adapters can be added to the same reaction. Adapters can be manipulated prior to combining with sample polynucleotides. For example, terminal phosphates can be added or removed.

Where adapter oligonucleotides are used, the adapter oligonucleotides can contain one or more of a variety of sequence elements, including but not limited to, one or more amplification primer annealing sequences or complements thereof, one or more sequencing primer annealing sequences or complements thereof, one or more barcode sequences, one or more common sequences shared among multiple different adapters or subsets of different adapters, one or more restriction enzyme recognition sites, one or more overhangs complementary to one or more target polynucleotide overhangs, one or more probe binding sites (e.g. for attachment to a sequencing platform, such as a flow cell for massive parallel sequencing, such as flow cells as developed by Illumina, Inc.), one or more random or near-random sequences (e.g. one or more nucleotides selected at random from a set of two or more different nucleotides at one or more positions, with each of the different nucleotides selected at one or more positions represented in a pool of adapters comprising the random sequence), and combinations thereof. In some cases, the adapters may be used to purify those circles that contain the adapters, for example by using beads (particularly magnetic beads for ease of handling) that are coated with oligonucleotides comprising a complementary sequence to the adapter, that can “capture” the closed circles with the correct adapters by hybridization thereto, wash away those circles that do not contain the adapters and any unligated components, and then release the captured circles from the beads. In addition, in some cases, the complex of the hybridized capture probe and the target circle can be directly used to generate concatemers, such as by direct rolling circle amplification (RCA). In some embodiments, the adapters in the circles can also be used as a sequencing primer. Two or more sequence elements can be non-adjacent to one another (e.g. separated by one or more nucleotides), adjacent to one another, partially overlapping, or completely overlapping. For example, an amplification primer annealing sequence can also serve as a sequencing primer annealing sequence. Sequence elements can be located at or near the 3′ end, at or near the 5′ end, or in the interior of the adapter oligonucleotide. A sequence element may be of any suitable length, such as about or less than about 3, 4, 5, 6, 7, 8, 9, 10, 15, 20, 25, 30, 35, 40, 45, 50 or more nucleotides in length. Adapter oligonucleotides can have any suitable length, at least sufficient to accommodate the one or more sequence elements of which they are comprised. In some embodiments, adapters are about or less than about 10, 15, 20, 25, 30, 35, 40, 45, 50, 55, 60, 65, 70, 75, 80, 90, 100, 200, or more nucleotides in length. In some embodiments, an adapter oligonucleotide is in the range of about 12 to 40 nucleotides in length, such as about 15 to 35 nucleotides in length.

In some embodiments, the adapter oligonucleotides joined to fragmented polynucleotides from one sample comprise one or more sequences common to all adapter oligonucleotides and a barcode that is unique to the adapters joined to polynucleotides of that particular sample, such that the barcode sequence can be used to distinguish polynucleotides originating from one sample or adapter joining reaction from polynucleotides originating from another sample or adapter joining reaction. In some embodiments, an adapter oligonucleotide comprises a 5′ overhang, a 3′ overhang, or both that is complementary to one or more target polynucleotide overhangs. Complementary overhangs can be one or more nucleotides in length, including but not limited to 1, 2, 3, 4, 5, 6, 7, 8, 9, 10, 11, 12, 13, 14, 15, or more nucleotides in length. Complementary overhangs may comprise a fixed sequence. Complementary overhangs of an adapter oligonucleotide may comprise a random sequence of one or more nucleotides, such that one or more nucleotides are selected at random from a set of two or more different nucleotides at one or more positions, with each of the different nucleotides selected at one or more positions represented in a pool of adapters with complementary overhangs comprising the random sequence. In some embodiments, an adapter overhang is complementary to a target polynucleotide overhang produced by restriction endonuclease digestion. In some embodiments, an adapter overhang consists of an adenine or a thymine.

A variety of methods for circularizing polynucleotides are available. In some embodiments, circularization comprises an enzymatic reaction, such as use of a ligase (e.g., an RNA or DNA ligase). A variety of ligases are available, including, but not limited to, Circligase™ (Epicentre; Madison, WI), RNA ligase, T4 RNA Ligase 1 (ssRNA Ligase, which works on both DNA and RNA). In addition, T4 DNA ligase can also ligate ssDNA if no dsDNA templates are present, although this is generally a slow reaction. Other non-limiting examples of ligases include NAD-dependent ligases including Taq DNA ligase, Thermus filiformis DNA ligase, Escherichia coli DNA ligase, Tth DNA ligase, Thermus scotoductus DNA ligase (I and II), thermostable ligase, Ampligase thermostable DNA ligase, VanC-type ligase, 9° N DNA Ligase, Tsp DNA ligase, and novel ligases discovered by bioprospecting; ATP-dependent ligases including T4 RNA ligase, T4 DNA ligase, T3 DNA ligase, T7 DNA ligase, Pfu DNA ligase, DNA ligase 1, DNA ligase III, DNA ligase IV, and novel ligases discovered by bioprospecting; and wild-type, mutant isoforms, and genetically engineered variants thereof. Where self-joining is desired, the concentration of polynucleotides and enzyme can be adjusted to facilitate the formation of intramolecular circles rather than intermolecular structures. Reaction temperatures and times can be adjusted as well. In some embodiments, 60° C. is used to facilitate intramolecular circles. In some embodiments, reaction times are between 12-16 hours. Reaction conditions may be those specified by the manufacturer of the selected enzyme. In some embodiments, an exonuclease step can be included to digest any unligated nucleic acids after the circularization reaction. That is, closed circles do not contain a free 5′ or 3′ end, and thus the introduction of a 5′ or 3′ exonuclease will not digest the closed circles but will digest the unligated components. This may find particular use in multiplex systems.

In general, joining ends of a polynucleotide to one-another to form a circular polynucleotide (either directly, or with one or more intermediate adapter oligonucleotides) produces a junction having a junction sequence. Where the 5′ end and 3′ end of a polynucleotide are joined via an adapter polynucleotide, the term “junction” can refer to a junction between the polynucleotide and the adapter (e.g. one of the 5′ end junction or the 3′ end junction), or to the junction between the 5′ end and the 3′ end of the polynucleotide as formed by and including the adapter polynucleotide. Where the 5′ end and the 3′ end of a polynucleotide are joined without an intervening adapter (e.g. the 5′ end and 3′ end of a single-stranded DNA), the term “junction” refers to the point at which these two ends are joined. A junction may be identified by the sequence of nucleotides comprising the junction (also referred to as the “junction sequence”). In some embodiments, samples comprise polynucleotides having a mixture of ends formed by natural degradation processes (such as cell lysis, cell death, and other processes by which DNA is released from a cell to its surrounding environment in which it may be further degraded, such as in cell-free polynucleotides, such as cell-free DNA and cell-free RNA), fragmentation that is a byproduct of sample processing (such as fixing, staining, and/or storage procedures), and fragmentation by methods that cleave DNA without restriction to specific target sequences (e.g. mechanical fragmentation, such as by sonication; non-sequence specific nuclease treatment, such as Dnase I, fragmentase). Where samples comprise polynucleotides having a mixture of ends, the likelihood that two polynucleotides will have the same 5′ end or 3′ end is low, and the likelihood that two polynucleotides will independently have both the same 5′ end and 3′ end is extremely low. Accordingly, in some embodiments, junctions may be used to distinguish different polynucleotides, even where the two polynucleotides comprise a portion having the same target sequence. Where polynucleotide ends are joined without an intervening adapter, a junction sequence may be identified by alignment to a reference sequence. For example, where the order of two component sequences appears to be reversed with respect to the reference sequence, the point at which the reversal appears to occur may be an indication of a junction at that point. Where polynucleotide ends are joined via one or more adapter sequences, a junction may be identified by proximity to the known adapter sequence, or by alignment as above if a sequencing read is of sufficient length to obtain sequence from both the 5′ and 3′ ends of the circularized polynucleotide. In some embodiments, the formation of a particular junction is a sufficiently rare event such that it is unique among the circularized polynucleotides of a sample.

### Methods of Sequencing

According to some embodiments of methods of detecting a tumor nucleic acid provided herein, linear and/or circularized polynucleotides (or amplification products thereof, which may have optionally been enriched) are subjected to a sequencing reaction to generate sequencing reads. Sequencing depth is chosen based on what is needed for the sample being sequenced. In some cases, sequencing is low depth or fewer reads or reads per molecule, used interchangeably herein. In some cases, sequencing is high depth or more reads or reads per molecule, used interchangeably herein. Sequencing reads produced by such methods may be used in accordance with other methods disclosed herein. A variety of sequencing methodologies are available, particularly high-throughput sequencing methodologies. Examples include, without limitation, sequencing systems manufactured by Illumina (sequencing systems such as HiSeq® and MiSeq®), Life Technologies (Ion Torrent®, SOLiD®, etc.), Roche's 454 Life Sciences systems, Pacific Biosciences systems, Oxford Nanopore Technologies, nanoball sequencing, sequencing by hybridization, polymerized colony (POLONY) sequencing, nanogrid rolling circle sequencing (ROLONY), etc. In some embodiments, sequencing comprises use of HiSeq® and MiSeq® systems to produce reads of about or more than about 50, 75, 100, 125, 150, 175, 200, 250, 300, or more nucleotides in length. In some embodiments, sequencing comprises a sequencing by synthesis process, where individual nucleotides are identified iteratively, as they are added to the growing primer extension product. Pyrosequencing is an example of a sequence by synthesis process that identifies the incorporation of a nucleotide by assaying the resulting synthesis mixture for the presence of by-products of the sequencing reaction, namely pyrophosphate. In particular, a primer/template/polymerase complex is contacted with a single type of nucleotide. If that nucleotide is incorporated, the polymerization reaction cleaves the nucleoside triphosphate between the a and R phosphates of the triphosphate chain, releasing pyrophosphate. The presence of released pyrophosphate is then identified using a chemiluminescent enzyme reporter system that converts the pyrophosphate, with AMP, into ATP, then measures ATP using a luciferase enzyme to produce measurable light signals. Where light is detected, the base is Incorporated, where no light is detected, the base is not incorporated. Following appropriate washing steps, the various bases are cyclically contacted with the complex to sequentially identify subsequent bases in the template sequence. See, e.g., U.S. Pat. No. 6,210,891.

In related sequencing processes, the primer/template/polymerase complex is immobilized upon a substrate and the complex is contacted with labeled nucleotides. The immobilization of the complex may be through the primer sequence, the template sequence and/or the polymerase enzyme, and may be covalent or noncovalent. For example, immobilization of the complex can be via a linkage between the polymerase or the primer and the substrate surface. In alternate configurations, the nucleotides are provided with and without removable terminator groups. Upon incorporation, the label is coupled with the complex and is thus detectable. In the case of terminator bearing nucleotides, all four different nucleotides, bearing individually identifiable labels, are contacted with the complex. Incorporation of the labeled nucleotide arrests extension, by virtue of the presence of the terminator, and adds the label to the complex, allowing identification of the incorporated nucleotide. The label and terminator are then removed from the incorporated nucleotide, and following appropriate washing steps, the process is repeated. In the case of non-terminated nucleotides, a single type of labeled nucleotide is added to the complex to determine whether it will be incorporated, as with pyrosequencing. Following removal of the label group on the nucleotide and appropriate washing steps, the various different nucleotides are cycled through the reaction mixture in the same process. See, e.g., U.S. Pat. No. 6,833,246, incorporated herein by reference in its entirety for all purposes. For example, the Illumina Genome Analyzer System is based on technology described in WO 98/44151, wherein DNA molecules are bound to a sequencing platform (flow cell) via an anchor probe binding site (otherwise referred to as a flow cell binding site) and amplified in situ on a glass slide. A solid surface on which DNA molecules are amplified typically comprise a plurality of first and second bound oligonucleotides, the first complementary to a sequence near or at one end of a target polynucleotide and the second complementary to a sequence near or at the other end of a target polynucleotide. This arrangement permits bridge amplification, such as described in US20140121116. The DNA molecules are then annealed to a sequencing primer and sequenced in parallel base-by-base using a reversible terminator approach. Hybridization of a sequencing primer may be preceded by cleavage of one strand of a double-stranded bridge polynucleotide at a cleavage site in one of the bound oligonucleotides anchoring the bridge, thus leaving one single strand not bound to the solid substrate that may be removed by denaturing, and the other strand bound and available for hybridization to a sequencing primer. Typically, the Illumina Genome Analyzer System utilizes flow-cells with 8 channels, generating sequencing reads of 18 to 36 bases in length, generating >1.3 Gbp of high quality data per run (see www.illumina.com).

In yet a further sequence by synthesis process, the incorporation of differently labeled nucleotides is observed in real time as template dependent synthesis is carried out. An individual immobilized primer/template/polymerase complex may be observed as fluorescently labeled nucleotides are incorporated, permitting real time identification of each added base as it is added. In this process, label groups may be attached to a portion of the nucleotide that is cleaved during incorporation. For example, by attaching the label group to a portion of the phosphate chain removed during incorporation, i.e., a β,γ, or other terminal phosphate group on a nucleoside polyphosphate, the label is not incorporated into the nascent strand, and instead, natural DNA is produced. Observation of individual molecules may involve the optical confinement of the complex within a very small illumination volume. By optically confining the complex, a monitored region may be created, in which randomly diffusing nucleotides may be present for a very short period of time, while incorporated nucleotides may be retained within the observation volume for longer as they are being incorporated. This may result in a characteristic signal associated with the incorporation event, which is also characterized by a signal profile that is characteristic of the base being added. Interacting label components, such as fluorescent resonant energy transfer (FRET) dye pairs, may be provided with the polymerase or other portion of the complex and the incorporating nucleotide, such that the incorporation event puts the labeling components in interactive proximity, and a characteristic signal results, that is again, also characteristic of the base being incorporated (See, e.g., U.S. Pat. Nos. 6,917,726, 7,033,764, 7,052,847, 7,056,676, 7,170,050, 7,361,466, and 7,416,844; and US 20070134128, each of which is entirely incorporated herein by reference).

In some embodiments, the nucleic acids in the sample can be sequenced by ligation. This method typically uses a DNA ligase enzyme to identify the target sequence, for example, as used in the polony method and in the SOLiD technology (Applied Biosystems, now Invitrogen). In general, a pool of all possible oligonucleotides of a fixed length is provided, labeled according to the sequenced position. Oligonucleotides are annealed and ligated; the preferential ligation by DNA ligase for matching sequences results in a signal corresponding to the complementary sequence at that position.

Sequencing methods of the present disclosure may provide information useful for various applications, such as, for example, identifying a disease (e.g., cancer) in a subject or determining that the subject is at risk of having (or developing) the disease. Sequencing may provide a sequence of a polymorphic region. Sequencing may provide a length of a polynucleotide, such as a DNA (e.g., cfDNA). Further, sequencing may provide a sequence of a breakpoint or end of a DNA, such as a cfDNA. Sequencing may provide a sequence of a border of a protein binding site or a border of a Dnase hypersensitive site.

### Samples

In some embodiments of the various methods described herein, the sample is from a subject. A subject may be any animal, including but not limited to, a cow, a pig, a mouse, a rat, a chicken, a cat, a dog, etc., and is usually a mammal, such as a human. Sample polynucleotides are often isolated from a cell-free sample from a subject, such as a tissue sample, bodily fluid sample, or organ sample, including, for example, blood sample, or fluid sample containing nucleic acids (e.g., saliva). In some cases, the sample is treated to remove cells, or polynucleotides are isolated without a cellular extractions step (e.g., to isolate cell-free polynucleotides, such as cell-free DNA). Other examples of sample sources include those from blood, urine, feces, nares, the lungs, the gut, other bodily fluids or excretions, materials derived therefrom, or combinations thereof. In some embodiments, the sample is a blood sample or a portion thereof (e.g., blood plasma or serum). Serum and plasma may be of particular interest, due to the relative enrichment for tumor DNA associated with the higher rate of malignant cell death among such tissues. In some embodiments, a sample from a single individual is divided into multiple separate samples (e.g., 2, 3, 4, 5, 6, 7, 8, 9, 10, or more separate samples) that are subjected to methods of the disclosure independently, such as analysis in duplicate, triplicate, quadruplicate, or more. Where a sample is from a subject, the reference sequence may also be derived from the subject, such as a consensus sequence from the sample under analysis or the sequence of polynucleotides from another sample or tissue of the same subject. For example, a blood sample may be analyzed for cfDNA mutations, while cellular DNA from another sample (e.g. buccal or skin sample) is analyzed to determine the reference sequence.

Polynucleotides may be extracted from a sample according to any suitable method. A variety of kits are available for extraction of polynucleotides, selection of which may depend on the type of sample, or the type of nucleic acid to be isolated. Examples of extraction methods are provided herein, such as those described with respect to any of the various aspects disclosed herein. In one example, the sample may be a blood sample, such as a sample collected in an EDTA tube (e.g., BD Vacutainer). Plasma can be separated from the peripheral blood cells by centrifugation (e.g., 10 minutes at 1900×g at 4° C.). Plasma separation performed in this way on a 6 mL blood sample will typically yield 2.5 to 3 mL of plasma. Circulating cell-free DNA can be extracted from a plasma sample, such as by using a QIAmp Circulating Nucleic Acid Kit (Qiagene), according the manufacturer's protocol. DNA may then be quantified (e.g., on an Agilent 2100 Bioanalyzer with High Sensitivity DNA kit (Agilent)). As an example, yield of circulating DNA from such a plasma sample from a healthy person may range from 1 ng to 10 ng per mL of plasma, with significantly more in disease (e.g., cancer) patient samples.

In some embodiments, the plurality of polynucleotides comprises cell-free polynucleotides, such as cell-free DNA (cfDNA), cell-free RNA (cfRNA), circulating tumor DNA (ctDNA), or circulating tumor RNA (ctRNA). Cell-free DNA circulates in both healthy and diseased individuals. Cell-free RNA circulates in both healthy and diseased individuals. cfDNA from tumors (ctDNA) is not confined to any specific cancer type but appears to be a common finding across different malignancies. According to some measurements, the free circulating DNA concentration in plasma is about 14-18 ng/ml in control subjects and about 180-318 ng/ml in patients with neoplasia. Apoptotic and necrotic cell death contribute to cell-free circulating DNA in bodily fluids. For example, significantly increased circulating DNA levels have been observed in plasma of prostate cancer patients and other prostate diseases, such as Benign Prostate Hyperplasia and Prostatitis. In addition, circulating tumor DNA is present in fluids originating from the organs where the primary tumor occurs. Thus, breast cancer detection can be achieved in ductal lavages; colorectal cancer detection in stool; lung cancer detection in sputum, and prostate cancer detection in urine or ejaculate. Cell-free DNA may be obtained from a variety of sources. One common source is blood samples of a subject. However, cfDNA or other fragmented DNA may be derived from a variety of other sources. For example, urine and stool samples can be a source of cfDNA, including ctDNA. Cell-free RNA may be obtained from a variety of sources.

In some embodiments, polynucleotides are subjected to subsequent steps (e.g., circularization and amplification) without an extraction step, and/or without a purification step. For example, a fluid sample may be treated to remove cells without an extraction step to produce a purified liquid sample and a cell sample, followed by isolation of DNA from the purified fluid sample. A variety of procedures for isolation of polynucleotides are available, such as by precipitation or non-specific binding to a substrate followed by washing the substrate to release bound polynucleotides. Where polynucleotides are isolated from a sample without a cellular extraction step, polynucleotides will largely be extracellular or “cell-free” polynucleotides. For example, cell-free polynucleotides may include cell-free DNA (also called “circulating” DNA). In some embodiments, the circulating DNA is circulating tumor DNA (ctDNA) from tumor cells, such as from a body fluid or excretion (e.g., blood sample). Cell-free polynucleotides may include cell-free RNA (also called “circulating” RNA). In some embodiments, the circulating RNA is circulating tumor RNA (ctRNA) from tumor cells. Tumors may show apoptosis or necrosis, such that tumor nucleic acids are released into the body, including the blood stream of a subject, through a variety of mechanisms, in different forms and at different levels. Typically, the size of the ctDNA can range between higher concentrations of smaller fragments, generally 70 to 200 nucleotides in length, to lower concentrations of large fragments of up to thousands kilobases.

### Cancer

Methods of detecting a tumor nucleic acid provided herein, in some cases comprise staging of a cancer. Staging of cancer is dependent on cancer type where each cancer type has its own classification system. Examples of cancer staging or classification systems are described in more detail below.

TABLE 1
Colon Cancer Primary Tumor (T)

| | |
|---|---|
| TX | Primary tumor cannot be assessed |
| T0 | No evidence of primary tumor |
| Tis | Carcinoma in situ: intraepithelial or intramucosal carcinoma |
| | (involvement of lamina propria with no extension through the |
| | muscularis mucosa) |
| T1 | Tumor invades submucosa (through the muscularis mucosa but |
| | not into the muscularis propria) |
| T2 | Tumor invades muscularis propria |
| T3 | Tumor invades through the muscularis propria into the |
| | pericolorectal tissues |
| T4 | Tumor invades the visceral peritoneum or invades or adheres to |
| | adjacent organ or structure |
| T4a | Tumor invades through the visceral peritoneum (including gross |
| | perforation of the bowel through tumor and continuous invasion |
| | of tumor through areas of inflammation to the surface of the |
| | visceral peritoneum) |
| T4b | Tumor directly invades or is adherent to other organs or structures |

Colon Cancer Regional Lymph Notes (N)

| | |
|---|---|
| NX | Regional lymph nodes cannot be assessed |
| N0 | No regional lymph node metastasis |
| N1 | Metastasis in 1-3 regional lymph nodes (tumor in lymph nodes |
| | measuring ≥0.2 mm) or any number of tumor deposits are present |
| | and all identifiable nodes are negative |
| N1a | Metastasis in 1 regional lymph node |
| N1b | Metastasis in 2-3 regional lymph nodes |
| N1c | Tumor deposit(s) in the subserosa, mesentery, or |
| | nonperitonealized, pericolic, or perirectal/mesorectal tissues |
| | without regional nodal metastasis |
| N2 | Metastasis in 4 or more lymph nodes |
| N2a | Metastasis in 4-6 regional lymph nodes |
| N2b | Metastasis in 7 or more regional lymph nodes |

Colon Cancer Distant Metastasis (M)

| | |
|---|---|
| M0 | No distant metastasis by imaging or other studies, no evidence of |
| | tumor in distant sites or organs. (This category is not assigned by |
| | pathologists.) |
| M1 | Metastasis to one or more distant sites or organs or peritoneal |
| | metastasis |
| M1a | Metastasis confined to 1 organ or site (e.g., liver, lung, ovary, |
| | nonregional node) without peritoneal metastasis |
| M1b | Metastasis to two or more sites or organs without peritoneal |
| | metastasis |
| M1c | Metastasis to the peritoneal surface alone or with other site or |
| | organ metastases |

TABLE 2
Colon Cancer Anatomic stage/prognostic groups

| Stage | T | N | M | Dukes | MAC |
|---|---|---|---|---|---|
| 0 | Tis | N0 | M0 | — | — |
| I | T1 | N0 | M0 | A | A |
| | T2 | N0 | M0 | A | B1 |
| IIA | T3 | N0 | M0 | B | B2 |
| IIB | T4a | N0 | M0 | B | B2 |
| IIC | T4b | N0 | M0 | B | B3 |
| IIIA | T1 − T2 | N1/N1c | M0 | C | C1 |
| | T1 | N2a | M0 | C | C1 |
| IIIB | T3 − T4a | N1/N1c | M0 | C | C2 |
| | T2 − T3 | N2a | M0 | C | C1/C2 |
| | T1 − T2 | N2b | M0 | C | C1 |
| IIIC | T4a | N2a | M0 | C | C2 |
| | T3 − T4a | N2b | M0 | C | C2 |
| | T4b | N1-N2 | M0 | C | C3 |
| IVA | Any T | Any N | M1a | — | — |
| IVB | Any T | Any N | M1b | — | — |
| IVC | Any T | Any N | M1c | — | — |

TABLE 3
Malignant Melanoma Primary Tumor (T)

| | |
|---|---|
| TX | Primary tumor cannot be assessed (i.e., Curettage melanoma) |
| T0 | No evidence of primary tumor |
| Tis | Melanoma in situ |
| T1 | Thickness ≤1.0 mm |
| | T1a: <0.8 mm without ulceration |
| | T1b: <0.8 mm with ulceration, or 0.8-1.0 mm with or without |
| | ulceration |
| T2 | Thickness >1.0-2.0 mm |
| | T2a: Without ulceration |
| | T2b: With ulceration |
| T3 | Thickness >2.0-4.0 mm |
| | T3a: Without ulceration |
| | T3b: With ulceration |
| T4 | Thickness >4.0 mm |
| | T4a: Without ulceration |
| | T4b: With ulceration |

Malignant Melanoma Regional Lymph Notes (N)

| | |
|---|---|
| NX | Regional lymph nodes cannot be assessed |
| N0 | No regional metastasis detected |
| N1 | One tumor-involved lymph node or in-transit, satellite, and/or |
| | microsatellite metastases with no tumor-involved nodes |
| | N1a: One clinically occult (i.e., detected by sentinel lymph node |
| | biopsy [SLNB]; no in-transit, satellite, or microsatellite |
| | metastases |
| | N1b: One clinically detected; no in-transit, satellite, or |
| | microsatellite metastases |
| | N1c: No regional lymph node disease; in-transit, satellite, and/or |
| | microsatellite metastases found |
| N2 | Two or three tumor-involved nodes; or in-transit, satellite, or |
| | microsatellite metastases |
| | N2a: Two or three clinically occult (i.e., detected by SLNB); no |
| | in-transit, satellite, or microsatellite metastases |
| | N2b: Two or three clinically detected; no in-transit, satellite, or |
| | microsatellite metastases |
| | N2c: One clinically occult or clinically detected; in-transit, |
| | satellite, and/or microsatellite metastases found |
| N3 | ≥4 tumor-involved nodes or in -transit, satellite, and/or |
| | microsatellite metastases with ≥2 tumor-involved nodes or any |
| | number of matted nodes without or with in-transit, satellite, and/or |
| | microsatellite metastases |
| | N3a: ≥4 clinically occult (i.e., detected by SLNB); no in-transit, |
| | satellite, or microsatellite metastases |
| | N3b: ≥4, at least one of which was clinically detected, or presence |
| | of any matted nodes; no in-transit, satellite, or microsatellite |
| | metastases |
| | N3c: ≥2 clinically occult or clinically detected and/or presence of |
| | any matted nodes, with presence of in-transit, satellite, and/or |
| | microsatellite metastases |

Malignant Melanoma Distant Metastasis (M)

| | |
|---|---|
| M0 | No detectable evidence of distant metastases |
| M1a | Metastases to skin, soft tissue (including muscle), and/or |
| | nonregional lymph nodes |
| M1b | Lung metastasis, with or without M1a involvement |
| M1c | Distant metastasis to non-central nervous system (CNS) |
| | visceral sites with or without M1a or M1b involvement |
| M1d | Distant metastasis to CNS, with or without M1a or M1b |
| | involvement |

TABLE 4
Malignant Melanoma Anatomic stage/prognostic groups

| Stage | T | N | M |
|---|---|---|---|
| 0 | Tis | N0 | M0 |
| IA | T1a | N0 | M0 |
| IB | T1b | N0 | M0 |
| | T2a | N0 | M0 |
| IIA | T2b | N0 | M0 |
| | T3a | N0 | M0 |
| IIB | T3b | N0 | M0 |
| | T4a | N0 | M0 |
| IIC | T4b | N0 | M0 |
| III | Any T, Tis | N1, N2, or N3 | M0 |
| IV | Any T | Any N | M1 |

TABLE 5
Hepatocellular Carcinoma Primary tumor (T)

| | |
|---|---|
| TX | Primary tumor cannot be assessed |
| T0 | No evidence of primary tumor |
| T1 | Solitary tumor 2 cm without vascular invasion |
| T1a | Solitary tumor <2 cm |
| T1b | Solitary tumor >2 cm without vascular invasion |
| T2 | Solitary tumor >2 cm with vascular invasion; or multiple tumors, |
| | non >5 cm |
| T3 | Multiple tumors, at least one of which is >5 cm |
| T4 | Single tumor or tumors of any size involving a major branch of |
| | the portal vein or hepatic vein, or tumor(s) with direct invasion |
| | of adjacent organs other than the gallbladder or with perforation |
| | of visceral peritoneum |

Hepatocellular Carcinoma Regional Lymph Nodes (N)

| | |
|---|---|
| NX | Regional lymph node(s) cannot be assessed |
| N0 | No regional lymph node metastasis |
| N1 | Regional lymph node metastasis |

Hepatocellular Carcinoma Distant Metastasis (M)

| | |
|---|---|
| M0 | No distant metastasis |
| M1 | Distant metastasis |

TABLE 6
Hepatocellular Carcinoma Anatomic stage/prognostic groups

| Stage | T | N | M |
|---|---|---|---|
| IA | T1a | N0 | M0 |
| IB | T1b | N0 | M0 |
| II | T2 | N0 | M0 |
| IIIA | T3 | N0 | M0 |
| IIIB | T4 | N0 | M0 |
| IVA | Any T | N1 | M0 |
| IVB | Any T | Any N | M1 |

TABLE 7
Hepatocellular Carcinoma Histologic grade

| | |
|---|---|
| GX | Grade cannot be accessed |
| G1 | Well differentiated |
| G2 | Moderately differentiated |
| G3 | Poorly differentiated |
| G4 | Undifferentiated |

TABLE 8
Barcelona-Clinic Liver Cancer staging system

| | Performance | | Okuda | |
|---|---|---|---|---|
| Stage | Status | Tumor Stage | Stage | Liver function |
| A: Early HCC | | | | |
| A1 | 0 | Single, <5 cm | I | No portal hypertension, |
| | | | | normal bilirubin |
| A2 | 0 | Single, <5 cm | I | Portal hypertension, |
| | | | | normal bilirubin |
| A3 | 0 | Single, <5 cm | I | Portal hypertension, |
| | | | | normal bilirubin |
| A4 | 0 | 3 tumors, <3 cm | I-II | Child-Pugh A-B |
| Stage B: | 0 | Large, multinodular | I-II | Child-Pugh A-B |
| Intermediate HCC | | | | |
| Stage C: | 1-2 | Vascular invasion or | I-II | Child-Pugh A-B |
| Advanced HCC | | extrahepatic spread | | |
| Stage D: | 3-4 | Any | I-II | Child-Pugh C |
| End-Stage HCC | | | | |

TABLE 9
Ishak Fibrosis score

| Architectural Change | Score |
|---|---|
| No fibrosis | 0 |
| Fibrous expansion of some portal areas, with or | 1 |
| without short fibrous septa | |
| Fibrous expansion of most portal areas, with or | 2 |
| without short fibrous septa | |
| Fibrous expansion of portal areas with occasional | 3 |
| portal-to-portal bridging | |
| Fibrous expansion of portal areas with marked | 4 |
| bridging as well as portal-central | |
| Marked bridging (portal-to-portal and/or portal- | 5 |
| central) with occasional nodule (incomplete | |
| cirrhosis) | |
| Cirrhosis, probable or definite | 6 |

TABLE 10
Gastric Cancer Primary tumor (T)

| | |
|---|---|
| TX | Primary tumor cannot be assessed |
| T0 | No evidence of primary tumor |
| Tis | Carcinoma in situ: intraepithelial tumor without |
| | invasion of the lamina propria |
| T1 | Tumor invades lamina propria, muscularis mucosae, or submucosa |
| T1a | Tumor invades lamina propria or muscularis mucosae |
| T1b | Tumor invades submucosa |
| T2 | Tumor invades muscularis propria |
| T3 | Tumor penetrates subserosal connective tissue without |
| | invasion of visceral peritoneum or adjacent structures. |
| T4 | Tumor invades serosa (visceral peritoneum) or adjacent structures |
| T4a | Tumor invades serosa (visceral peritoneum) |
| T4b | Tumor invades adjacent structures |

Regional Lymph Nodes (N)

| | |
|---|---|
| NX | Regional lymph node(s) cannot be assessed |
| N0 | No regional lymph node metastasis |
| N1 | Metastasis in 1-2 regional lymph nodes |
| N2 | Metastasis in 3-6 regional lymph nodes |
| N3 | Metastasis in seven or more regional lymph nodes |
| N3a | Metastasis in 7-15 regional lymph nodes |
| N3b | Metastasis in 16 or more regional lymph nodes |

Distant Metastasis (M)

| | |
|---|---|
| M0 | No distant metastasis |
| M1 | Distant metastasis |

TABLE 11
Gastric Cancer Clinical stage/prognostic groups (cTNM)

| Stage | T | N | M |
|---|---|---|---|
| 0 | Tis | N0 | M0 |
| I | T1 | N0 | M0 |
| | T2 | N0 | M0 |
| IIA | T1 | N1, N2, N3 | M0 |
| | T2 | N1, N2, N3 | M0 |
| IIB | T3 | N0 | M0 |
| | T4 | N0 | M0 |
| III | T | N1, N2, N3 | M0 |
| | T4a | N1, N2, N3 | M0 |
| IVA | Any T | Any N | M0 |
| IVB | Any T | Any N | M1 |

TABLE 12
Gastric Cancer Pathological stage (pTNM)

| Stage | T | N | M |
|---|---|---|---|
| 0 | Tis | N0 | M0 |
| I | T1 | N0 | M0 |
| | T1 | N1 | M0 |
| IB | T2 | N0 | M0 |
| | T1 | N2 | M0 |
| II A | T2 | N1 | M0 |
| | T3 | N0 | M0 |
| | T1 | N3 | M0 |
| | T2 | N2 | M0 |

TABLE 13
Gastric Cancer Post-neoadjuvant therapy
staging and overall survival (ypTNM)

| | | | | 3-year | 5-year |
|---|---|---|---|---|---|
| | | | | survival | survival |
| Stage | T | N | M | (%) | (%) |
| I | T1, T2 | N0 | M0 | 81.4 | 76.5 |
| | T1 | N1 | M0 | | |
| | T1 | N2, N3 | M0 | | |
| | T2 | N1, N2 | M0 | | |
| II | T3 | N0, N1 | M0 | 54.8 | 46.3 |
| | T4a | N0 | M0 | | |
| | T2 | N3 | M0 | | |
| | T3 | M2, N3 | M0 | | |
| III | T4a | N1, N2, N3 | M0 | | |
| | T4b | N0, N1, N2, N3 | M0 | 28.8 | 18.3 |
| IV | Any T | Any N | M1 | 10.2 | 5.7 |

TABLE 14
Esophageal Cancer Primary tumor (T)

| | |
|---|---|
| TX | Primary tumor cannot be assessed |
| T0 | No evidence of primary tumor |
| Tis | High-grade dysplasia, * defined as malignant cells confined |
| | by the basement membrane |
| T1 | Tumor invades lamina propria, muscularis mucosae, or submucosa |
| T1a | Tumor invades lamina propria or muscularis mucosae |
| T1b | Tumor invades submucosa |
| T2 | Tumor invades muscularis propria |
| T3 | Tumor invades adventitia |
| T4 | Tumor invades adjacent structures |
| T4a | Resectable tumor invading pleura, pericardium, azygos vein, |
| | diaphragm or peritoneum |
| T4b | Unresectable tumor invading other adjacent structures, such |
| | as the aorta, vertebral body, and trachea |

Esophageal Cancer Regional Lymph Nodes (N)

| | |
|---|---|
| NX | Regional lymph node(s) cannot be assessed |
| N0 | No regional lymph node metastasis |
| N1 | Metastasis in 1-2 regional lymph nodes |
| N2 | Metastasis in 3-6 regional lymph nodes |
| N3 | Metastasis in 7 or more regional lymph nodes |

Esophageal Cancer Distant Metastasis (M)

| | |
|---|---|
| M0 | No distant metastasis |
| M1 | Distant metastasis |

TABLE 15
Esophageal Cancer Histologic grade
Histologic grade (G)

| | |
|---|---|
| GX | Grade cannot be assessed - stage grouping as G1 |
| G1 | Well differentiated |
| G2 | Moderately differentiated |
| G3 | Poorly differentiated or undifferentiated* |

TABLE 16
Squamous cell carcinoma location

| | |
|---|---|
| X | Location unknown |
| Upper | Cervical esophagus to lower border of azygos vein |
| Middle | Lower border of azygos vein to lower border of inferior |
| | pulmonary vein |
| Lower | Lower border of inferior pulmonary vein to stomach, including |
| | gastroesophageal junction |

TABLE 17
Esophageal Cancer Clinical stage groups

| | | | |
|---|---|---|---|
| Stage Group | cT | cN | cM |

Squamous cell carcinoma

| | | | |
|---|---|---|---|
| 0 | Tis | N0 | M0 |
| I | T1 | N0-1 | M0 |
| | T2 | N0-1 | M0 |
| II | T3 | N0 | M0 |
| | T3 | N1 | M0 |
| III | T1-3 | N2 | M0 |
| | T4 | N0-2 | M0 |
| IVA | T1-4 | N3 | M0 |
| IVB | T1-4 | N0-3 | M1 |

Adenocarcinoma

| | | | |
|---|---|---|---|
| 0 | Tis | N0 | M0 |
| I | T1 | N0 | M0 |
| IIA | T1 | N1 | M0 |
| IIB | T2 | N0 | M0 |
| | T2 | N1 | M0 |
| III | T3-4a | N0-1 | M0 |
| | T1-4a | N2 | M0 |
| IVA | T4b | N0-2 | M0 |
| | T1-4 | N3 | M0 |
| IVB | T1-4 | N0-3 | M1 |

TABLE 18
Pathologic stage groups

| | | | | | |
|---|---|---|---|---|---|
| Stage Group | pT | pN | pM | Grade | Location |

Squamous cell carcinoma

| | | | | | |
|---|---|---|---|---|---|
| 0 | Tis | N0 | M0 | N/A | Any |
| IA | T1a | N0 | M0 | G-1, X | Any |
| | T1b | N0 | M0 | G1-3, X | Any |
| IB | T1a | N0 | M0 | G2-3 | Any |
| | T2 | N0 | M0 | G1 | Any |
| | T2 | N0 | M0 | G2-3, X | Any |
| IIA | T3 | N0 | M0 | Any | Lower |
| | T34 | N0 | M0 | G1 | Upper/middle |
| | T3 | N0 | M0 | G2-3 | Upper/middle |
| | T3 | N0 | M0 | GX | Any |
| IIB | T3 | N0 | M0 | Any | X |
| | T1 | N1 | M0 | Any | Any |
| IIIA | T1 | N2 | M0 | Any | Any |
| | T2 | N1 | M0 | Any | Any |
| | T4a | N0-1 | M0 | Any | Any |
| IIIB | T3 | N1 | M0 | Any | Any |
| | T2-3 | N2 | M0 | Any | Any |
| | T4a | N2 | M0 | Any | Any |
| IVA | T4b | N0-2 | M0 | Any | Any |
| | T1-4 | N3 | M0 | Any | Any |
| IVB | T1-4 | N0-3 | M1 | Any | Any |

Adenocarcinoma

| | | | | |
|---|---|---|---|---|
| 0 | Tis | N0 | M0 | N/A |
| IA | T1a | N0 | M0 | G1, X |
| IB | T1a | N0 | M0 | G2 |
| | T1b | N0 | M0 | G1-2, X |
| | T1 | N0 | M0 | G3 |
| IC | T2 | N0 | M0 | G1-2 |
| IIA | T2 | N0 | M0 | G3, X |
| | T1 | N1 | M0 | Any |
| IIB | T3 | N0 | M0 | Any |
| | T1 | N2 | M0 | Any |
| IIIA | T2 | N1 | M0 | Any |
| | T4a | N0-1 | M0 | Any |
| IIIB | T3 | N1 | M0 | Any |
| | T2-3 | N2 | M0 | Any |
| IVA | T4a | N2 | M0 | Any |
| | T4b | N0-2 | M0 | Any |
| | T1-4 | N3 | M0 | Any |
| | R1-4 | N0-3 | M1 | Any |

TABLE 19
Postneoadjuvant therapy staging

| | | | |
|---|---|---|---|
| Stage Group | yPT | ypN | ypM |

Squamous cell carcinoma

| | | | |
|---|---|---|---|
| I | T0-2 | N0 | M0 |
| II | T3 | N0 | M0 |
| IIIA | T0-2 | N1 | M0 |
| | T4a | N0 | M0 |
| IIIB | T3 | N1 | M0 |
| | T0-3 | N2 | M0 |
| | T4a | N1-2, X | M0 |
| IVA | T4b | N0-2 | M0 |
| | T1-4 | N3 | M0 |
| IVB | T1-4 | N0-3 | M1 |

TABLE 20

| | | |
|---|---|---|
| | FIGO | |
| TNM | stages | Surgical-pathologic findings |

Endometrial Cancer Primary Tumor (T)

| | | |
|---|---|---|
| TX | | Primary tumor cannot be assessed |
| T0 | | No evidence of primary tumor |
| Tis | | Carcinoma in situ (preinvasive carcinoma) |
| T1 | I | Tumor confined to corpus uteri |
| T1a | IA | Tumor linked to endometrium or invades less than one |
| | | half of the myometrium |
| T1b | IB | Tumor invades one half or more of the myometrium |
| T2 | II | Tumor invades stromal connective tissue of the cervix |
| | | but does not extend beyond uterus** |
| T3a | IIIA | Tumor involves serosa and/or adnexa (direct extension or |
| | | metastasis) |
| T3b | IIIB | Vaginal involvement (direct extension or metastasis) or |
| | | parametrial involvement |
| | IIIC | Metastases to pelvic and/or para-aortic lymph nodes |
| | IV | Tumor invades bladder mucosa and/or bowel mucosa, |
| | | and/or distant metastases |
| T4 | IVA | Tumor invades bladder mucosa and/or bowel mucosa |
| | | (bullous edema is not sufficient to classify a tumor |
| | | as T4) |

Endometrial Cancer Regional Lymph Nodes (N)

| | | |
|---|---|---|
| NX | | Regional lymph nodes cannot be assessed |
| N0 | | No regional lymph node metastasis |
| N1 | IIIC1 | Regional lymph node metastasis to pelvic lymph nodes |
| N2 | IIIC2 | Regional lymph node metastasis to para-aortic lymph |
| | | nodes, with or without positive pelvic lymph nodes |

Endometrial Cancer Distant Metastasis

| | |
|---|---|
| M0 | No distant metastasis |
| M1 | Distant metastasis (includes metastasis to inguinal lymph |
| | nodes, intraperitoneal M1 IVB disease, or lung, liver, or |
| | bone metastases; it excludes metastasis to para-aortic |
| | lymph nodes, vagina, pelvic serosa, or adnexa) |

TABLE 21
Non-Small Cell Lung Cancer Primary tumor (T)

| | |
|---|---|
| TX | Primary tumor cannot be assessed, or tumor is proven by the presence of malignant cells |
| | in sputum or bronchial washings but not visualized by imaging or bronchoscopy |
| T0 | No evidence of primary tumor |
| Tis | Carcinoma in situ |
| | Squamous cell carcinoma in situ (SCIS) |
| | Adenocarcinoma in situ (AIS): adenocarcinoma with pure lepidic pattern, ≤3 cm in |
| | greatest dimension |
| T1 | Tumor ≤3 cm in greatest dimension, surrounded by lung or visceral pleura, without |
| | bronchoscopic evidence of invasion more proximal than the lobar bronchus (i.e., not in the |
| | main bronchus) |
| T1mi | Minimally invasive adenocarcinoma: adenocarcinoma (≤3 cm in greatest dimension) with |
| | a predominantly lepidic pattern and ≤5 mm invasion in greatest dimension |
| T1a | Tumor ≤1 cm in greatest dimension. A superficial, spreading tumor of any size whose |
| | invasive component is limited to the bronchial wall and may extend proximal to the main |
| | bronchus also is classified as T1a, but those tumors are uncommon. |
| T1b | Tumor >1 cm but ≤2 cm in greatest dimension |
| T1c | Tumor >2 cm but ≤3 cm in greatest dimension |
| T2 | Tumor >3 cm but ≤5 cm or having any of the following features: |
| | Involves the main bronchus regardless of distance to the carina, but without |
| | involvement of the carina |
| | Invades visceral pleura (PL1 or PL2) |
| | Associated with atelectasis or obstructive pneumonitis extending to the hilar |
| | region, involving part or all of the lung |
| | T2 tumors with these features are classified as T2a if ≤ 4 cm or if the size cannot be |
| | determined and T2b if >4 cm but ≤5 cm |
| T2a | Tumor >3 cm but ≤4 cm in greatest dimension |
| T2b | Tumor >4 cm but ≤5 cm in greatest dimension |
| T3 | Tumor >5 cm but ≤7 cm in greatest dimension or directly invading any of the following: |
| | parietal pleural (PL3), chest wall (including superior sulcus tumors), phrenic nerve, |
| | parietal pericardium; or separate tumor nodule(s) in the same lobe as the primary |
| T4 | Tumor >7 cm or tumor of any size that invades one or more of the following: diaphragm, |
| | mediastinum, heart, great vessels, trachea, recurrent laryngeal nerve, esophagus, vertebral |
| | body, or carina; or separate tumor nodule(s) in an ipsilateral lobe different from that of the |
| | primary |

Non-Small Cell Lung Cancer Regional lymph nodes (N)

| | |
|---|---|
| NX | Regional lymph nodes cannot be assessed |
| N0 | No regional node metastasis |
| N1 | Metastasis in ipsilateral peribronchial and/or ipsilateral hilar lymph nodes and |
| | intrapulmonary nodes, including involvement by direct extension |
| N2 | Metastasis in ipsilateral mediastinal and/or subcarinal lymph node(s) |
| N3c | Metastasis in the contralateral mediastinal, contralateral hilar, ipsilateral or contralateral |
| | scalene, or supraclavicular lymph node(s) |

Non-Small Cell Lung Cancer Distant metastasis (M)

| | |
|---|---|
| M0 | No distant metastasis |
| M1 | Distant metastasis |
| M1a | Separate tumor nodule(s) in a contralateral lobe tumor; tumor with pleural or pericardial |
| | nodules or malignant pleural or pericardial effusion. Most pleural (pericardial) effusion |
| | with lung cancer are a result of the tumor. In a few patients, however, multiple |
| | microscopic examinations of pleural (pericardial) fluid are negative for tumor, and the |
| | fluid is nonbloody and not an exudate. If these elements and clinical judgment dictate that |
| | the effusion is not related to the tumor, the effusion should be excluded as a staging |
| | descriptor. |
| M1b | Single extrathoracic metastasis in a single organ and involvement of a single nonregional |
| | node |
| M1c | Multiple extrathoracic metastases in a single organ or in multiple organs |

TABLE 22
Non-Small Cell Lung Cancer Anatomic stage/prognostic groups

| Stage | T | N | M |
|---|---|---|---|
| 0 | Tis | N0 | M0 |
| | T1mi | N0 | M0 |
| IA1 | T1a | N0 | M0 |
| IA2 | T1b | N0 | M0 |
| IA3 | T1c | N0 | M0 |
| IB | T2a | N0 | M0 |
| IIA | T2b | N0 | M0 |
| IIB | T1a | N1 | M0 |
| | T1b | N1 | M0 |
| | T1c | N1 | M0 |
| | T2a | N1 | M0 |
| | T2b | N1 | M0 |
| | T3 | N0 | M0 |
| | T1a | N2 | M0 |
| | T1b | N2 | M0 |
| | T1c | N2 | M0 |
| | T2a | N2 | M0 |
| IIIA | T2b | N2 | M0 |
| | T3 | N1 | M0 |
| | T4 | N0 | M0 |
| | T4 | N1 | M0 |
| IIIB | T1a | N3 | M0 |
| | T1b | N3 | M0 |
| | T1c | N3 | M0 |
| | T2a | N3 | M0 |
| | T2b | N3 | M0 |
| | T3 | N2 | M0 |
| | T4 | N2 | M0 |
| | T3 | N3 | M0 |
| IIIC | T4 | N3 | M0 |
| IVA | T Any | N Any | M1a |
| T Any | | N Any | M1b |
| IVB | T Any | N Any | M1c |

TABLE 23
Small Cell Lung Cancer Primary tumor (T)

| | |
|---|---|
| TX | Primary tumor cannot be assessed, or tumor is proven by the presence of malignant cells in |
| | sputum or bronchial washings but not visualized by imaging or bronchoscopy |
| TC | No evidence of primary tumor |
| Tis | Carcinoma in situ |
| | Squamous cell carcinoma in situ (SCIS) |
| | Adenocarcinoma in situ (AIS): adenocarcinoma with pure lepidic pattern, ≤3 cm in |
| | greatest dimension |
| T1 | Tumor ≤3 cm in greatest dimension, surrounded by lung or visceral pleura, without |
| | bronchoscopic evidence of invasion more proximal than the lobar bronchus (i.e., not in the |
| | main bronchus) |
| T1mi | Minimally invasive adenocarcinoma: adenocarcinoma (≤3 cm in greatest dimension) with |
| | a predominantly lepidic pattern and ≤5 mm invasion in greatest dimension |
| T1a | Tumor ≤1 cm in greatest dimension. A superficial, spreading tumor of any size whose |
| | invasive component is limited to the bronchial wall and may extend proximal to the main |
| | bronchus also is classified as Tla, but those tumors are uncommon. |
| T1b | Tumor >1 cm but ≤2 cm in greatest dimension |
| T1c | Tumor >2 cm but ≤3 cm in greatest dimension |
| T2 | Tumor >3 cm but ≤5 cm or having any of the following features: |
| | Involves the main bronchus regardless of distance to the carina, but without |
| | involvement of the carina |
| | Invades visceral pleura (PL1 or PL2) |
| | Associated with atelectasis or obstructive pneumonitis extending to the hilar |
| | region, involving part or all of the lung |
| | T2 tumors with these features are classified as T2a if ≤4 cm or if the size cannot be |
| | determined and T2b if >4 cm but ≤5 cm |
| T2a | Tumor >3 cm but ≤4 cm in greatest dimension |
| T2b | Tumor >4 cm but ≤5 cm in greatest dimension |
| T3 | Tumor >5 cm but ≤7 cm in greatest dimension or directly invading any of the following: |
| | parietal pleural (PL3), chest wall (including superior sulcus tumors), phrenic nerve, |
| | parietal pericardium; or separate tumor nodule(s) in the same lobe as the primary |
| T4 | Tumor >7 cm or tumor of any size that invades one or more of the following: diaphragm, |
| | mediastinum, heart, great vessels, trachea, recurrent laryngeal nerve, esophagus, vertebral |
| | body, or carina; or separate tumor nodule(s) in an ipsilateral lobe different from that of the |
| | primary |

Small Cell Lung Cancer Regional lymph nodes (N)

| | |
|---|---|
| NX | Regional lymph nodes cannot be assessed |
| N0 | No regional lymph node metastasis |
| N1 | Metastasis to ipsilateral peribronchial and/or ipsilateral hilar lymph nodes and |
| | intrapulmonary nodes, including involvement by direct extension |
| N2 | Metastases in ipsilateral mediastinal and/or subcarinal lymph node(s) |
| N3 | Metastasis in contralateral mediastinal, contralateral hilar, ipsilateral or contralateral |
| | scalene, or supraclavicular lymph node(s) |

Small Cell Lung Cancer Distant metastasis (M)

| | |
|---|---|
| M0 | No distant metastasis |
| M1 | Distant metastases |
| M1a | Separate tumor nodule(s) in a contralateral lobe tumor; tumor with pleural or pericardial |
| | nodules or malignant pleural or pericardial effusion. Most pleural (pericardial) effusion |
| | with lung cancer are a result of the tumor. In a few patients, however, multiple microscopic |
| | examinations of pleural (pericardial) fluid are negative for tumor, and the fluid is |
| | nonbloody and not an exudate. If these elements and clinical judgment dictate that the |
| | effusion is not related to the tumor, the effusion should be excluded as a staging descriptor. |
| M1b | Single extrathoracic metastasis in a single organ and involvement of a single nonregional |
| | node |
| M1c | Multiple extrathoracic metastases in a single organ or in multiple organs |

TABLE 24
Small Cell Lung Cancer Anatomic stage/prognostic groups

| | | | |
|---|---|---|---|
| Stage | T | N | M |

Limited disease

| | | | |
|---|---|---|---|
| 0 | Tis | N0 | M0 |
| | T1mi | N0 | M0 |
| IA1 | T1a | N0 | M0 |
| IA2 | T1b | N0 | M0 |
| IA3 | T1c | N0 | M0 |
| IB | T2a | N0 | M0 |
| IIA | T2b | N0 | M0 |
| IIB | T1a | N1 | M0 |
| | T1b | N1 | M0 |
| | T1c | N1 | M0 |
| | T2a | N1 | M0 |
| | T2b | N1 | M0 |
| | T3 | N0 | M0 |
| | T1a | N2 | M0 |
| | T1b | N2 | M0 |
| | T1c | N2 | M0 |
| IIIA | T2a | N2 | M0 |
| | T2b | N2 | M0 |
| | T3 | N1 | M0 |
| | T4 | N0 | M0 |
| | T4 | N1 | M0 |
| IIIB | T1a | N3 | M0 |
| | T1b | N3 | M0 |
| | T1c | N3 | M0 |
| | T2a | N3 | M0 |
| | T2b | N3 | M0 |
| | T3 | N2 | M0 |
| | T4 | N2 | M0 |
| IIIC | T3 | N3 | M0 |

Extensive disease

| | | | |
|---|---|---|---|
| IVA | T Any | N Any | M1a |
| | T Any | N Any | M1b |
| IVB | T Any | N Any | M1c |

TABLE 25
Breast Cancer Primary tumor (T)

| | |
|---|---|
| TX | Primary tumor cannot be assessed |
| T0 | No evidence of primary tumor |
| Tis | Carcinoma in situ |
| Tis (DCIS) | Ductal carcinoma in situ |
| Tis | Paget disease of the nipple N0T associated with invasive carcinoma and/or carcinoma in |
| (Paget) | situ (DCIS) in the underlying breast parenchyma. Carcinomas in the breast parenchyma |
| | associated with Paget disease are categorized on the basis of the size and characteristics of |
| | the parenchymal disease, although the presence of Paget disease should still be noted |
| T1 | Tumor ≤20 mm in greatest dimension |
| T1mi | Tumor ≤1 mm in greatest dimension |
| T1a | Tumor >1 mm but ≤5 mm in greatest dimension (round any measurement >1.0-1.9 mm to |
| | 2 mm) |
| T1b | Tumor >5 mm but ≤10 mm in greatest dimension |
| T1c | Tumor >10 mm but ≤20 mm in greatest dimension |
| T2 | Tumor >20 mm but ≤50 mm in greatest dimension |
| T3 | Tumor >50 mm in greatest dimension |
| T4 | Tumor of any size with direct extension to the chest wall and/or to the skin (ulceration or |
| | skin nodules), not including invasion of dermis alone |
| T4a | Extension to chest wall, not including only pectoralis muscle adherence/invasion |
| T4b | Ulceration and/or ipsilateral satellite nodules and/or edema (including peaud'orange) of |
| | the skin, which do not meet the criteria for inflammatory carcinoma |
| T4c | Both T4a and T4b |
| T4d | Inflammatory carcinoma |

Breast Cancer Regional lymph nodes (N)
Clinical

| | |
|---|---|
| cNX | Regional lymph nodes cannot be assessed (e.g., previously removed) |
| cN0 | No regional lymph node metastasis (on imaging or clinical examination) |
| cN1 | Metastasis to movable ipsilateral level I, II axillary lymph node(s) |
| cN1mi | Micrometastases (approximately 200 cells, larger than 0.2 mm, but none larger than 2.0 |
| | mm) |
| cN2 | Metastases in ipsilateral level I, II axillary lymph nodes that are clinically fixed or matted; |
| | or in ipsilateral internal mammary nodes in the absence of clinically evident axillary |
| | lymph node metastases |
| cN2a | Metastases in ipsilateral level I, II axillary lymph nodes fixed to one another (matted) or to |
| | other structures |
| cN2b | Metastases only in ipsilateral internal mammary nodes and in the absence of axillary |
| | lymph node metastases |
| cN3 | Metastases in ipsilateral infraclavicular (level III axillary) lymph node(s), with or without |
| | level I, II axillary node involvement, or in ipsilateral internal mammary lymph node(s) |
| | with level I, II axillary lymph node metastasis; or metastases in ipsilateral supraclavicular |
| | lymph node(s), with or without axillary or internal mammary lymph node involvement |
| cN3a | Metastasis in ipsilateral infraclavicular lymph node(s) |
| cN3b | Metastasis in ipsilateral internal mammary lymph node(s) and axillary lymph node(s) |
| cN3c | Metastasis in ipsilateral supraclavicular lymph node(s) |

Breast Cancer Pathologic (pN)

| | |
|---|---|
| pNX | Regional lymph nodes cannot be assessed (for example, previously removed, or not |
| | removed for pathologic study) |
| pN0 | No regional lymph node metastasis identified histologically, or isolated tumor cell clusters |
| | (ITCs) only. Note: ITCs are defined as small clusters of cells ≤0.2 mm, or single tumor |
| | cells, or a cluster of <200 cells in a single histologic cross-section; ITCs may be detected |
| | by routine histology or by immunohistochemical (IHC) methods; nodes containing only |
| | ITCs are excluded from the total positive node count for purposes of N classification but |
| | should be included in the total number of nodes evaluated |
| pN0(i) | No regional lymph node metastases histologically, negative IHC |
| pN0(i+) | ITCs only in regional lymph node(s) |
| pN0(mol−) | No regional lymph node metastases histologically, negative molecular findings (reverse |
| | transcriptase polymerase chain reaction [RT-PCR]) |
| pN0(mol+) | Positive molecular findings by RT-PCR; no ITCs detected |
| pN1 | Micrometastases; or metastases in 1-3 axillary lymph nodes and/or in internal mammary |
| | nodes; and/or in clinically negative internal mammary nodes with micrometastases or |
| | macrometastases by sentinel lymph node biopsy |
| pN1mi | Micrometastases (200 cells, >0.2 mm but none >2.0 mm) |
| pN1a | Metastases in 1-3 axillary lymph nodes (at least 1 metastasis >2.0 mm) |
| pN1b | Metastases in ipsilateral internal mammary lymph nodes, excluding ITCs, detected by |
| | sentinel lymph node biopsy |
| pN1c | Metastases in 1-3 axillary lymph nodes and in internal mammary sentinel nodes (i.e., pN1a |
| | and pN1b combined) |
| pN2 | Metastases in 4-9 axillary lymph nodes; or positive ipsilateral internal mammary lymph |
| | nodes by imaging in the absence of axillary lymph node metastases |
| pN2a | Metastases in 4-9 axillary lymph nodes (at least 1 tumor deposit >2.0 mm) |
| pN2b | Clinically detected*1 metastases in internal mammary lymph nodes with or without |
| | microscopic confirmation; with pathologically negative axillary lymph nodes |
| pN3 | Metastases in ≥10 axillary lymph nodes; or in infraclavicular (level III axillary) lymph |
| | nodes; or positive ipsilateral internal mammary lymph nodes by imaging in the presence of |
| | one or more positive level I, II axillary lymph nodes; or in >3 axillary lymph nodes and |
| | micrometastases or macrometastases by sentinel lymph node biopsy in clinically negative |
| | ipsilateral internal mammary lymph nodes; or in ipsilateral supraclavicular lymph nodes |
| pN3a | Metastases in ≥10 axillary lymph nodes (at least 1 tumor deposit >2.0 mm); or metastases |
| | to the infraclavicular (level III axillary lymph) nodes |
| pN3b | pN1a or pN2a in the presence of cN2b (positive internal mammary nodes by imaging) or |
| | pN2a in the presence of pN1b |
| pN3c | Metastases in ipsilateral supraclavicular lymph nodes |

Breast Cancer Distant metastasis (M)

| | |
|---|---|
| M0 | No clinical or radiographic evidence of distant metastasis |
| cM0(i+) | No clinical or radiographic evidence of distant metastases in the presence of tumor cells or |
| | deposits no larger than 0.2 mm detected microscopically or by molecular techniques in |
| | circulating blood, bone marrow, or other nonregional nodal tissue in a patient without |
| | symptoms or signs of metastasis |
| cM1 | Distant metastases detected by clinical and radiographic approaches |
| pM1 | Any histologically proven metastases in distant organs; or if in non-regional nodes, |
| | metastases >0.2 mm |

TABLE 26
Breast Cancer Histologic grade (G)

| | |
|---|---|
| GX | Grade cannot be assessed |
| G1 | Low combined histologic grade (favorable) |
| G2 | Intermediate combined histologic grade (moderately favorable) |
| G3 | High combined histologic grade (unfavorable) |

TABLE 27
Breast Cancer Anatomic stage/prognostic groups

| Stage | T | N | M |
|---|---|---|---|
| 0 | Tis | N0 | M0 |
| IA | T1 | N0 | M0 |
| IB | T0 | N1mi | M0 |
| | T1 | N1mi | M0 |
| IIA | T0 | N1 | M0 |
| | T1 | N1 | M0 |
| | T2 | N0 | M0 |
| IIB | T2 | N1 | M0 |
| | T3 | N0 | M0 |
| IIIA | T0 | N2 | M0 |
| | T1 | N2 | M0 |
| | T2 | N2 | M0 |
| | T3 | N1 | M0 |
| | T3 | N2 | M0 |
| IIIB | T4 | N0 | M0 |
| | T4 | N1 | M0 |
| | T4 | N2 | M0 |
| IIIC | Any T | N3 | M0 |
| IV | Any T | Any N | M1 |

In aspects of methods of detecting a tumor nucleic acid provided herein. Examples of tumor nucleic acids that may be detected in accordance with a method disclosed herein include, without limitation, Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof.

### Computer Systems

The present disclosure provides computer systems that are programmed to implement methods of detecting a tumor nucleic acid. FIG. 3 shows a computer system 301 that is programmed or otherwise configured to detect a tumor nucleic acid. The computer system 301 can regulate various aspects of methods of detecting tumor nucleic acids of the present disclosure, such as, for example, detecting tumor nucleic acids in a cell-free nucleic acids using a low depth sequencing method. The computer system 301 can be an electronic device of a user or a computer system that is remotely located with respect to the electronic device. The electronic device can be a mobile electronic device.

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet. The network 330 in some cases is a telecommunication and/or data network. The network 330 can include one or more computer servers, which can enable distributed computing, such as cloud computing. The network 330, in some cases with the aid of the computer system 301, can implement a peer-to-peer network, which may enable devices coupled to the computer system 301 to behave as a client or a server.

The CPU 305 can execute a sequence of machine-readable instructions, which can be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 301 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user (e.g., a person wishing to detect tumor nucleic acids). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 can include or be in communication with an electronic display 335 that comprises a user interface (UI) 340 for providing, for example, sequencing results showing detection of tumor nucleic acids. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 305. The algorithm can, for example, detect tumor nucleic acids.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Detecting Tumor Nucleic Acids in a Cell-Free Sample

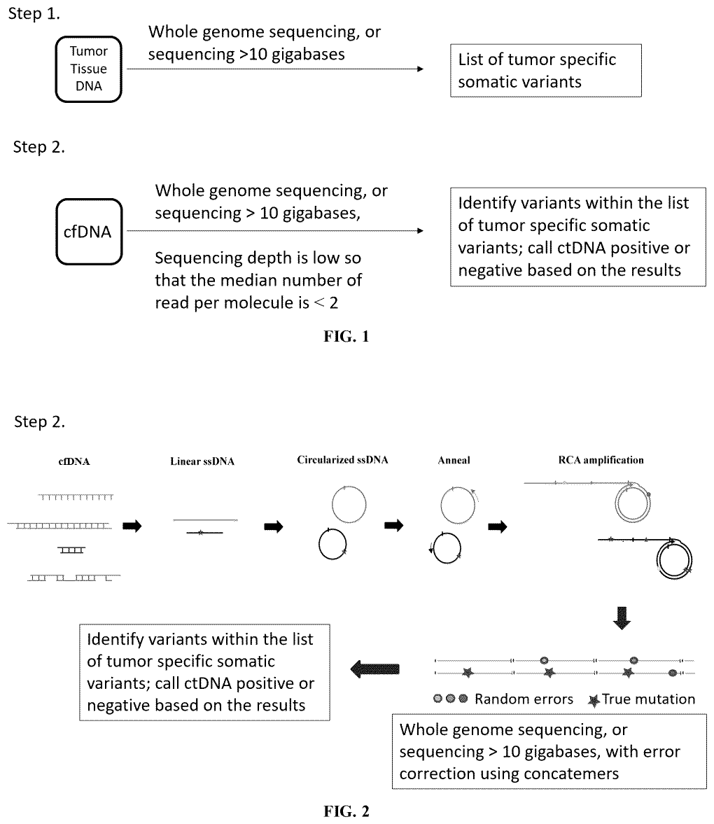

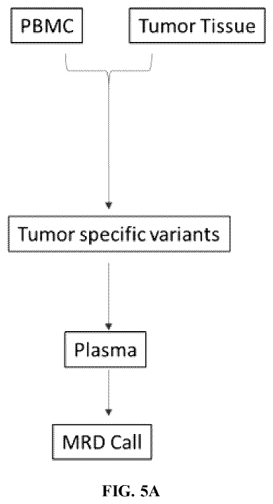

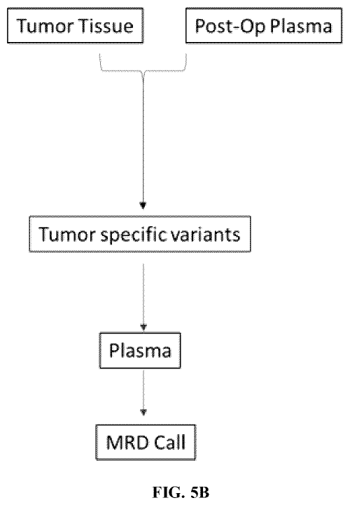

A method of tumor informed disease detection and monitoring is used to detect tumor nucleic acid in a sample. The method starts with conducting whole genome sequencing (e.g., greater than 10 gigabases) of DNA obtained from tumor tissue to identify a list of tumor specific somatic variants in a subject. The tumor specific variants can be identified by comparing sequences of the tumor DNA to sequences from healthy tissue DNA or from post-op plasma DNA. At the same time or a later time, cell-free DNA is obtained from a sample from the subject. The cell-free DNA is circularized and amplified using rolling circle amplification to obtain concatemer copies of the cell-free DNA that comprise two or more copies of the sequence of the cell-free DNA molecules. Whole genome sequencing is performed on the concatemers at low sequencing depth, for example less than two reads per molecule in order to determine whether the sample is positive or negative for tumor nucleic acids based on the presence or absence of the tumor specific variants. Determination of whether the sample is positive or negative for the tumor specific variants uses error correction, where the variant is only called when more than one occurrence of the variant is observed in the concatemer. The method is shown in FIG. 1, FIG. 2, and FIGS. 5A-5B.

### Example 2: Detecting Tumor Nucleic Acids with Genome Complexity Reduction

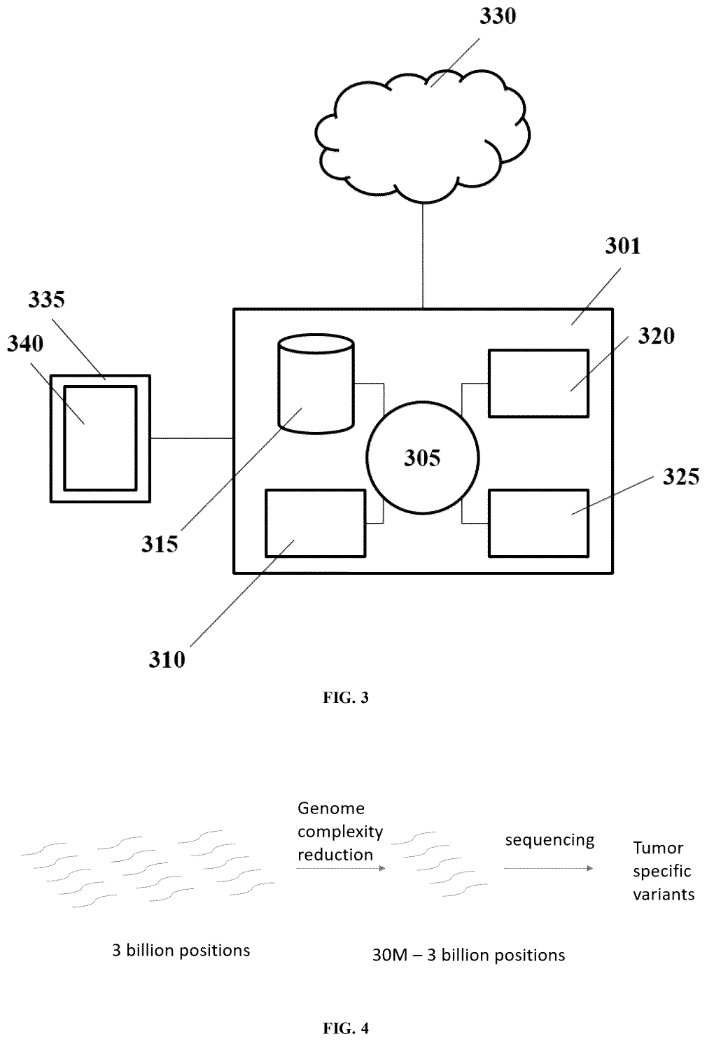

A method of tumor informed disease detection and monitoring is used to detect tumor nucleic acid in a sample. The method starts with reducing genome complexity to about 30 million positions from about 3 billion positions by using positive selection for target sequences on the tumor and normal nucleic acid sample (FIG. 4). Then whole genome sequencing is conducted to identify a list of tumor specific somatic variants in a subject. Cell-free DNA is also obtained from a sample from the subject. The cell-free DNA is circularized and amplified using rolling circle amplification to obtain concatemer copies of the cell-free DNA that comprise two or more copies of the sequence of the cell-free DNA molecules. Whole genome sequencing is performed on the amplified cell-free DNA at a low sequencing depth in order to determine whether the sample is positive or negative for tumor nucleic acids based on the presence or absence of the tumor specific variants. Determination of whether the sample is positive or negative for the tumor specific variants uses error correction, where the variant is only called when more than one occurrence of the variant is observed in the concatemer.

### Example 3: Negative Selection Workflow

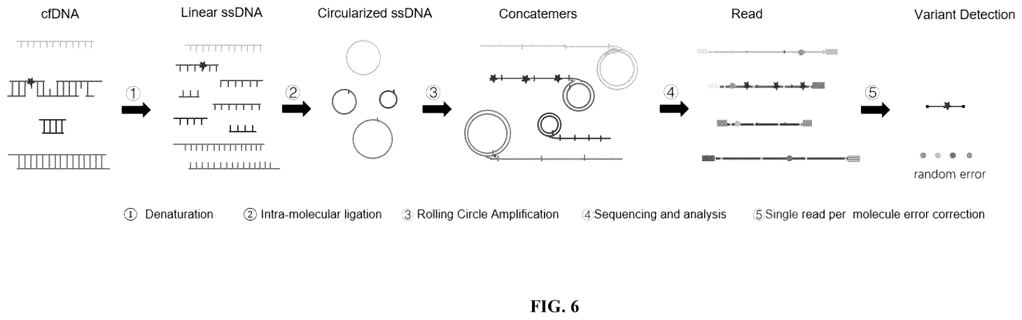

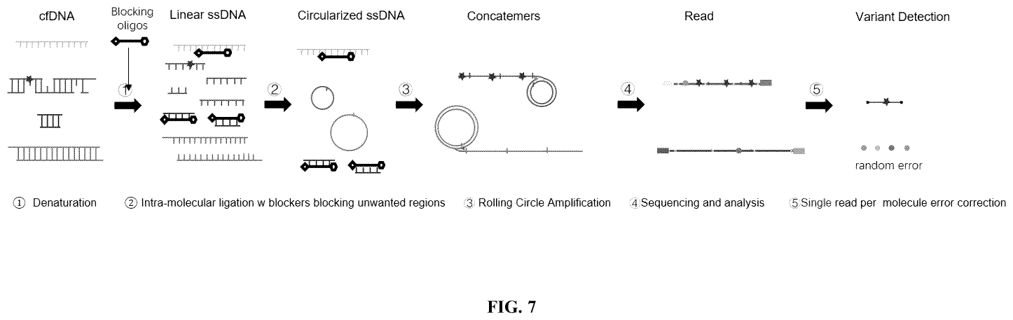

A nucleic acid sample, such as a cell-free DNA sample having sequences of interest and unwanted sequences is subjected to negative selection. The nucleic acids in the sample are subjected to a denaturation step and then blocking oligonucleotides having modified 5′ and/or 3′ ends are annealed to the unwanted sequences in the nucleic acid sample. The blocking oligonucleotides cannot ligate and cannot be extended with a polymerase. Therefore, only the single stranded nucleic acids in the sample will be subjected to circularization. Next the remaining linear nucleic acids that are bound to the blocking oligonucleotides are optionally degraded with a DNA exonuclease. The circularized nucleic acids are amplified by rolling circle amplification to generate concatemers. The concatemers are subjected to sequencing to obtain sequences of the nucleic acids and variants are detected based on their presence in one or more copy of the sequence in a concatemer. This method is shown in FIG. 7. The workflow without selection is shown in FIG. 6.

### Example 4: Positive Selection Workflow

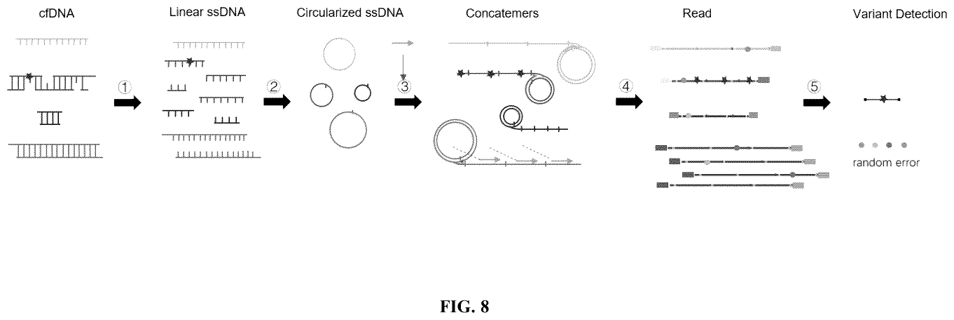

A nucleic acid sample, such as a cell-free DNA sample having sequences of interest and unwanted sequences is subjected to positive selection. The nucleic acids in the sample are subjected to a denaturation step subjected to circularization. The circularized nucleic acids are amplified by rolling circle amplification to generate concatemers using a combination of random primers and target specific primers resulting in enhanced amplification of sequences of interest. The concatemers are subjected to sequencing to obtain sequences of the nucleic acids and variants are detected based on their presence in one or more copy of the sequence in a concatemer. This method is shown in FIG. 8. The workflow without selection is shown in FIG. 6.

### Example 5: Ultra-Sensitive Circulating Tumor DNA Detection Through Whole Genome Sequencing with Single-Read Error Correction

Whole genome sequencing (WGS) of cell-free DNA (cfDNA) is useful for circulating tumor DNA (ctDNA) detection and the assessment of tumor burden. While the breadth of WGS may compensate for the scarcity of cfDNA, its sensitivity is limited by error rate. Described herein is AccuScan, which is an efficient cfDNA WGS technology that enables genome-wide error suppression at single read level achieving an error rate of 4.2×10⁻⁷, which is more than an order of magnitude lower than a read-centric de-noising method. When applied to molecular residual disease (MRD) detection, the method demonstrated analytical sensitivity down to 10⁻⁶ circulating tumor allele fraction (cTAF) at 99% sample level specificity. In colorectal cancer, AccuScan showed 90% landmark sensitivity for predicting relapse. It also proved robust MRD performance with esophageal cancer using samples collected as early as 1 week after surgery, and strong prognostic value for immunotherapy monitoring with melanoma patients. Overall, AccuScan provides a highly accurate WGS solution that enables ctDNA detection at ppm range without deep sequencing or personalized reagents.

Genome Wide Error Suppression Enables Detection of Ultra-Low ctDNA Levels

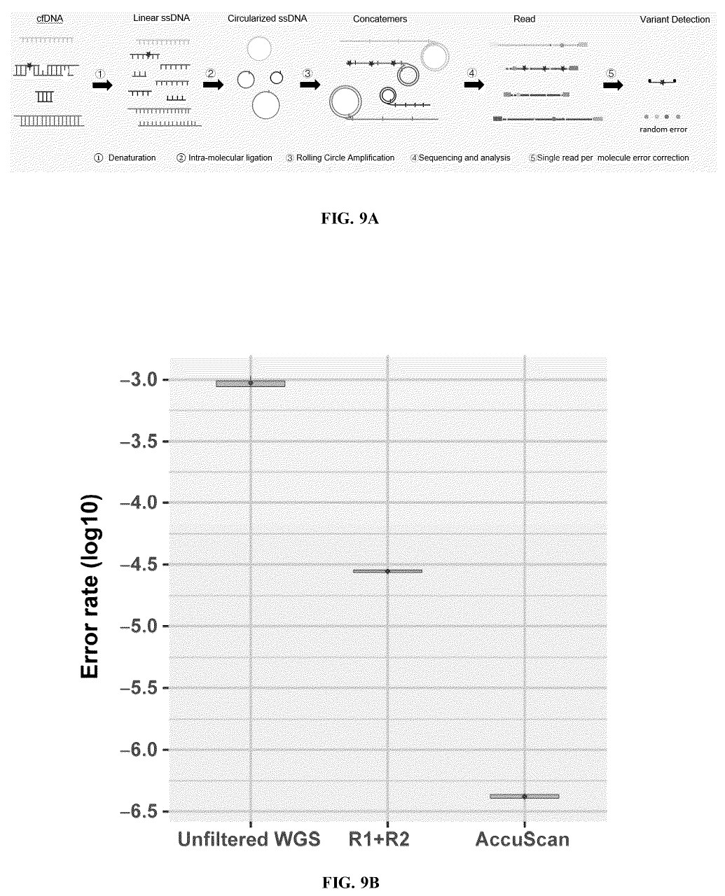

The AccuScan assay workflow (FIG. 9A) is optimized for low input cfDNA, efficiently capturing double strand, single strand DNA and nicked DNA in a sample. cfDNA is denatured and circularized through ligation, followed by whole-genome amplification using RCA, generating concatemer molecules containing multiple tandem copies of the original template. These concatemer products are sequenced using PE150 read length and aligned to the human reference genome. Sequences of each copy within a read pair are compared. A change from the reference that is consistent in all copies is a presumed variant; and a change that is inconsistent is likely to be PCR or sequencing errors and is removed. To assess the efficiency of error correction by AccuScan, the same cfDNA samples from healthy donors (N=3) were sequenced using both regular WGS and AccuScan WGS. FIG. 9B shows comparison of the measured error rates The observed overall error rate is ˜4.2×10⁻⁷ for AccuScan, and 3.3×10⁻⁴ using regular WGS with qscore filter, suggesting a ˜1000-fold noise reduction by concatemer error correction.

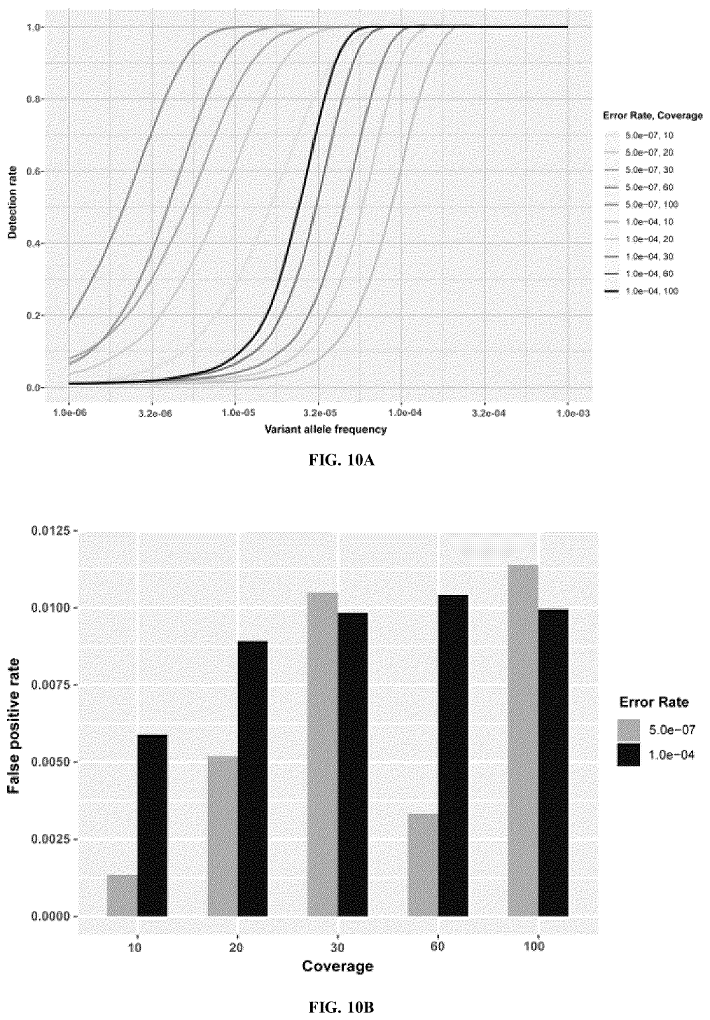

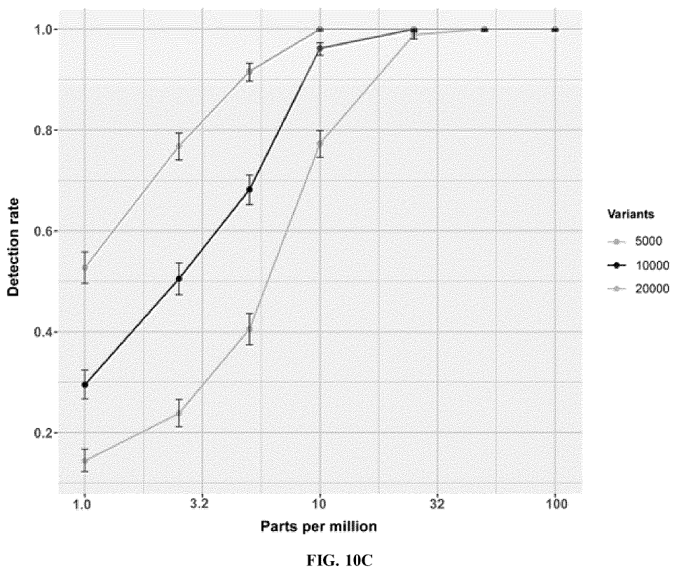

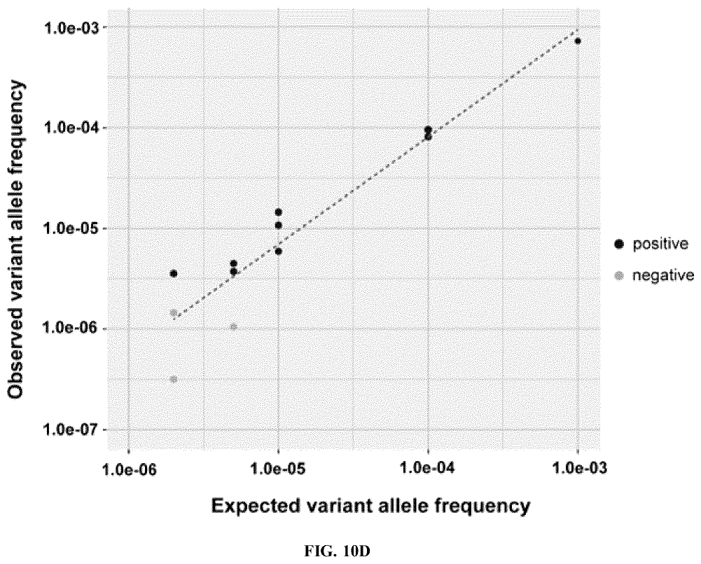

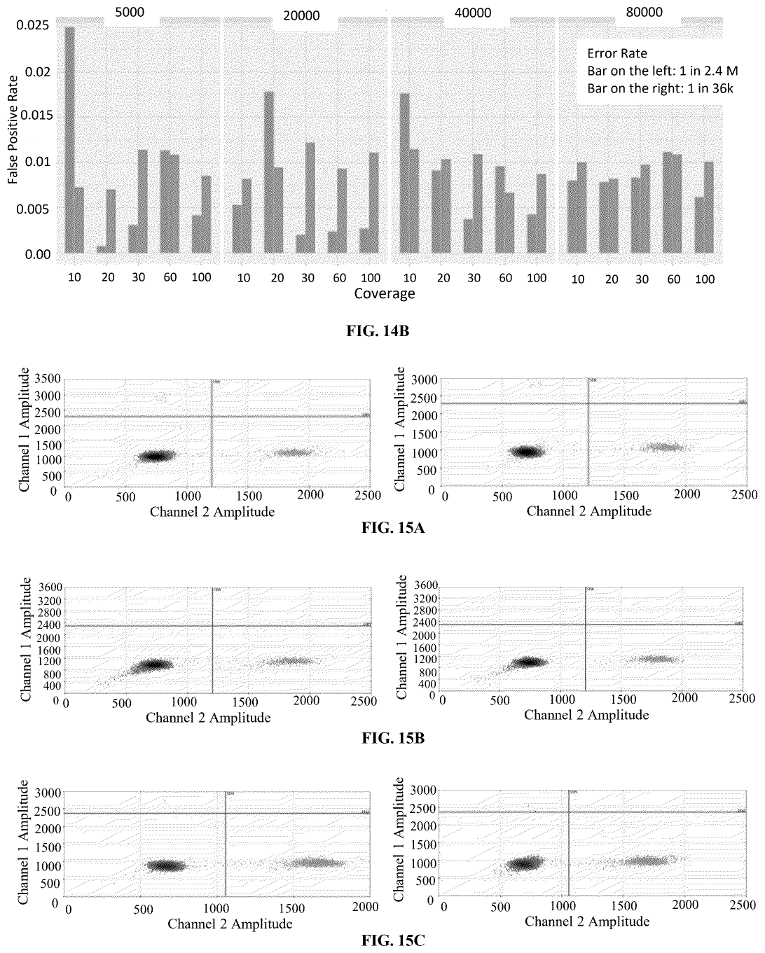

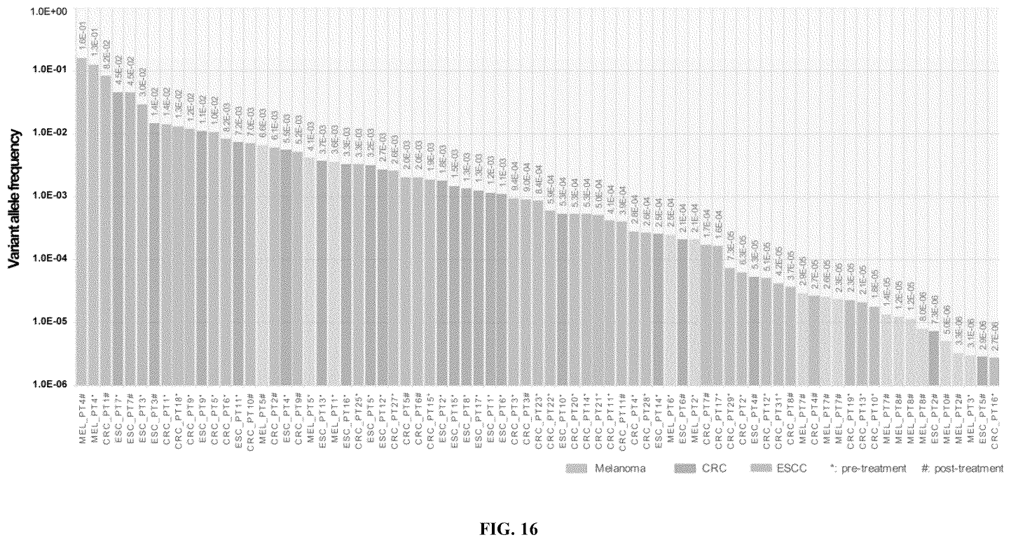

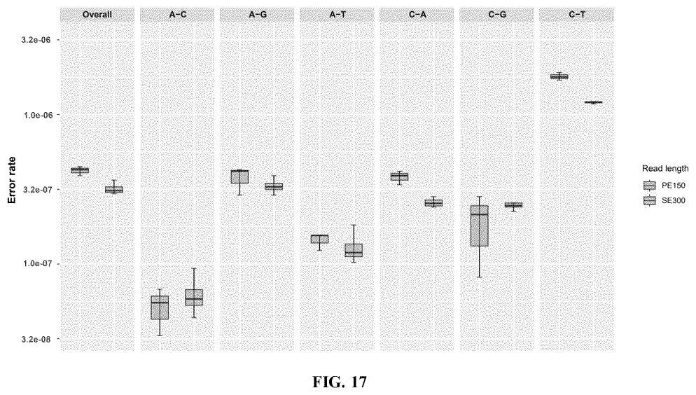

Simulations were performed to predict the impact of error rate on ctDNA detection under different cTAF, sequencing depth and number of markers (FIGS. 10A-10D, FIGS. 14A-14B). A statistical model that calculates the probability of observing expected base calls at specific marker loci is used to predict for the presence of ctDNA. Sensitivity is calculated as the number of positive predicted over the total number of simulations for each combination, under the nominal specificity setting of 99%.

FIG. 10A shows the sensitivity from simulations given 10000 markers with either 1×10⁻⁴ or 5×10⁷ error rate. Decreasing error rate or increasing sequencing depth both improve the detection rate. With an error rate of 5×10⁻⁷ and a 10× sequencing depth, there is a 95% detection rate (LOD95) at a cTAF of 4.5×10⁻⁵, but when the error rate is 1×10⁻⁴, 100× sequencing is required to achieve a similar LOD95 at the same cTAF. At 60× sequencing depth, a LOD95 of 1×10⁻⁵ is expected. FIG. 10B shows that the false positive rate with cTAF set to 0 remains under 1.2% across all conditions, which is consistent with the nominal specificity setting.

The analytical sensitivity of AccuScan was measured using healthy sample mixtures (FIG. 10C). cfDNA from three different healthy “test” donors was titrated independently into cfDNA from a different healthy “background” donor at 7 different concentrations ranging from 1×10⁻⁴ to 1×10⁻⁶. These 21 cfDNA mix samples were then sequenced to 60× using AccuScan with 10 ng input DNA per reaction and the ability to detect the “test” donor SNPs from background was assessed. Out of the over 100,000 SNVs at which each test and background sample pair differed randomly selected subsets of 5000, 10000, or 2000 SNVs (SNVs were selected to have a variant type profile similar to CRC tumors) were tested. This was repeated 1000 times per condition and MRD testing was run with 99% nominal specificity (Table 28). The observed specificities are >99% for 5000, 10000 or 20000 markers conditions. The observed sensitivity at 2.5×10⁻⁵ cTAF and above level is greater than 99% for all conditions tested. At 10 parts per million corresponding to 1×10⁻⁵ cTAF, the average detection rate of 5000 markers is 77%, 10000 marker tests showed an average sensitivity of 96%, and tests with 20000 markers maintained 100% sensitivity in all replicates.

TABLE 28
Specificity Results

| Variants | Nominal Specificity | Measured Specificity |
|---|---|---|
| 5000 | 99 | 99.4 |
| 10000 | 99 | 99.3 |
| 20000 | 99 | 99.2 |

The analytical sensitivity of AccuScan was further confirmed by mixing cfDNA from a melanoma patient with cfDNA from a healthy donor. The original cancer cfDNA sample had a cTAF of 1.1% as measured by ddPCR of a BRAF V600E mutation found in the primary tumor. Dilutions were made of 5 different expected frequencies from 1×10⁻³ to 2×10⁻⁶ and ddPCR was performed to confirm the BRAFV600E VAF of the 1×10⁻³ dilution. The diluted cancer samples were sequenced by AccuScan with 10 ng input per reaction. The observed detection rate is 100% for samples with cTAF of 1×10⁻³, 1×10⁻⁴ and 1×10⁻⁵, 67% (2/3) for 5×10⁻⁶ and 33% (1/3) for 2×10⁻⁶ (FIG. 10D). AccuScan sequencing of a negative control (cfDNA from a healthy donor) was negative in both replicates.

To measure the sample level specificity of AccuScan, tumor-specific variants from 60 different cancer patients were used, including CRC, ESCC and melanoma, randomly sampled 5K, 10K and 20K equivalent variants for testing the MRD call in mismatched patient plasma samples. 2000 random samplings of mismatched variants were done for each combination of variant count level and plasma sample. The average sample level specificity is computed as the fraction of MRD tests that are characterized with a negative MRD call. The observed values were similar to the nominal specificity, with 99.3%, 99.1%, and 98.9%, for 5K, 10K and 20K variant count levels, respectively. These results suggest that the AccuScan assay and analysis have the intended performance for patient plasmas using tumor variants.

### Identification of Tumor-Specific Variants Using a White Blood Cell (WBC) Free Workflow

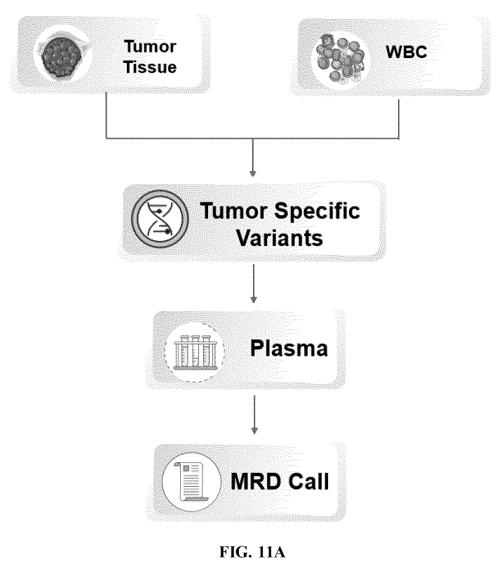

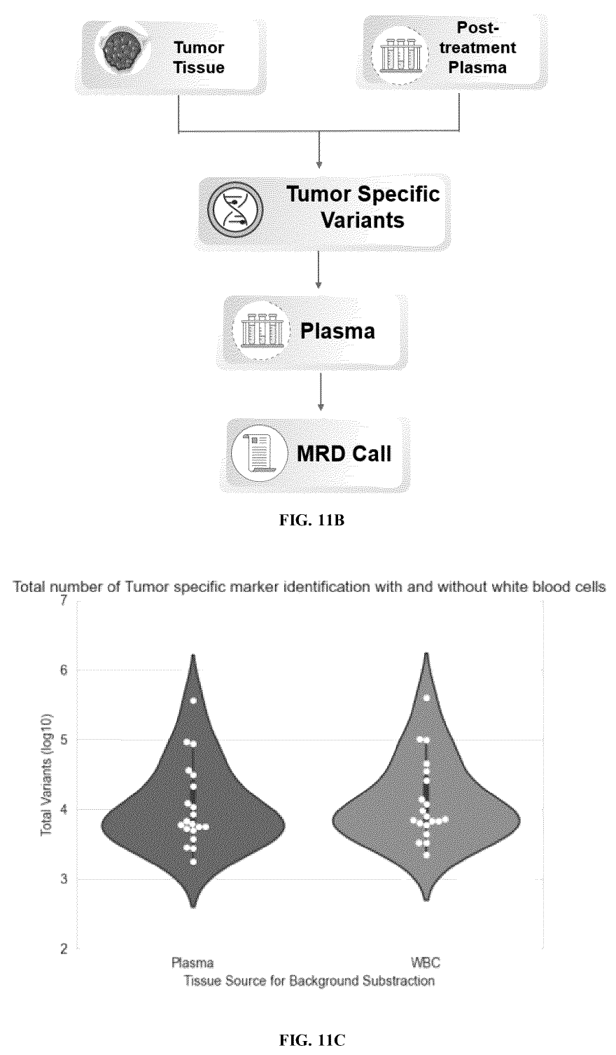

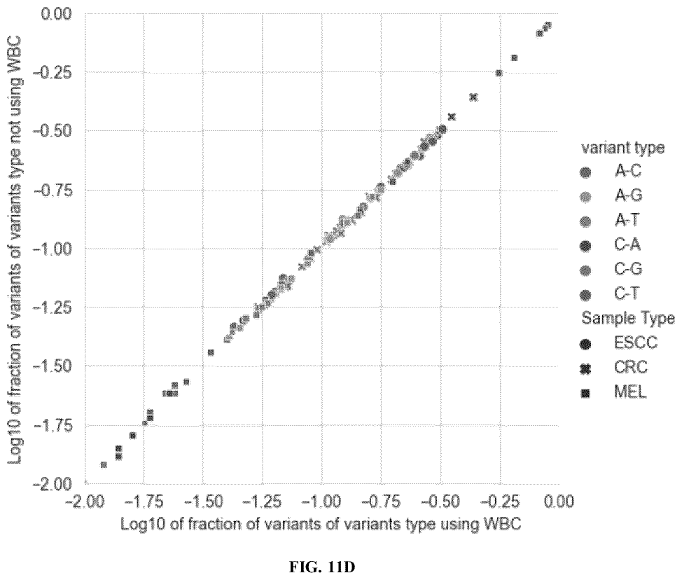

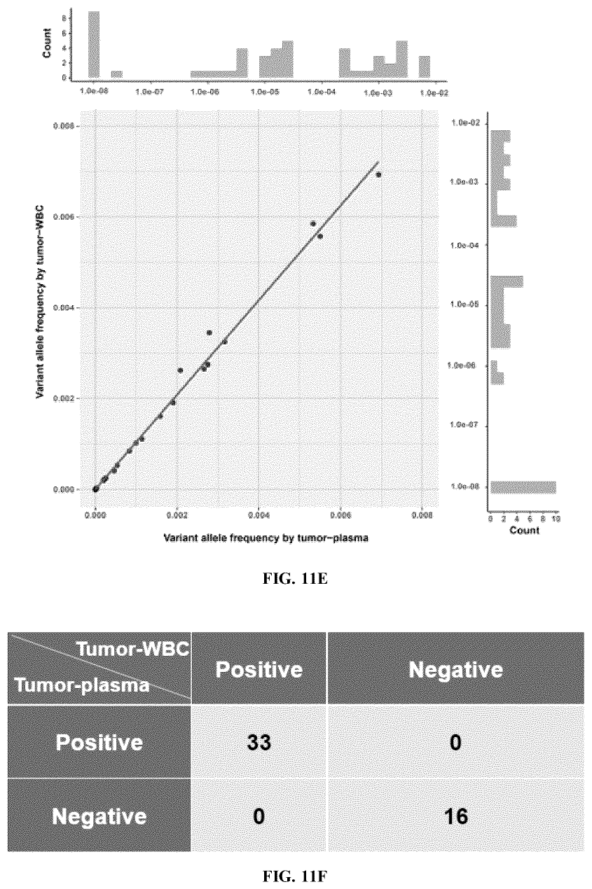

A tumor-informed MRD test uses tumor-specific variants as markers for tracking the disease. Sequencing of tumor tissues finds not only cancer mutations, but also germline SNPs and other types of variants such as clonal hematopoiesis of indeterminate potential (CHIP) variants, which will interfere MRD analysis. One common strategy for filtering non-cancer mutations is to remove variants found in the matching WBC from the same patient. However, this method requires extra sample processing and sequencing (FIG. 11A). To simplify the MRD workflow, the effect of skipping WBC sequencing and using information from the post-treatment plasma samples was investigated to remove germline and CHIP variants (FIG. 11B).

With 40× sequencing of a low-tumor-burden plasma sample, germline and CHIP mutations can be found at ≥2 molecules level, while tumor-specific variants will only be found at single molecule level. Hence, variants found with 2 or more molecules in the post-treatment plasmas can be removed from the tumor tissue sequencing result to obtain the list of tumor-specific mutations. To test the feasibility of this approach, the performance of tumor-WBC pair and tumor-plasma pairs were compared using matched tumor tissue, WBC and plasma samples collected from 20 cancer patient samples. The number of tumor specific variants and variant type profiles found by the two different workflows are shown in FIGS. 11C-11D. Overall, the number of mutations identified by both methods are very similar, as are the mutation profile of the variants identified. AccuScan MRD analysis of plasma samples (n=48) from these 20 patients returned identical MRD calls under either workflow (FIG. 11F) and the cTAF values are strongly correlated (R²=0.99, FIG. 11E). These results suggest that it is feasible to use post-treatment plasma in the place of WBC for tumor-specific variant identification.

### MRD Detection and Prognostic Value in Surgical Patients

Next, the performance of AccuScan for MRD detection in post-surgical gastrointestinal cancers, including 32 Colorectal Cancer (CRC) patients and 17 Esophageal Squamous Cell Carcinoma (ESCC) patients was evaluated.

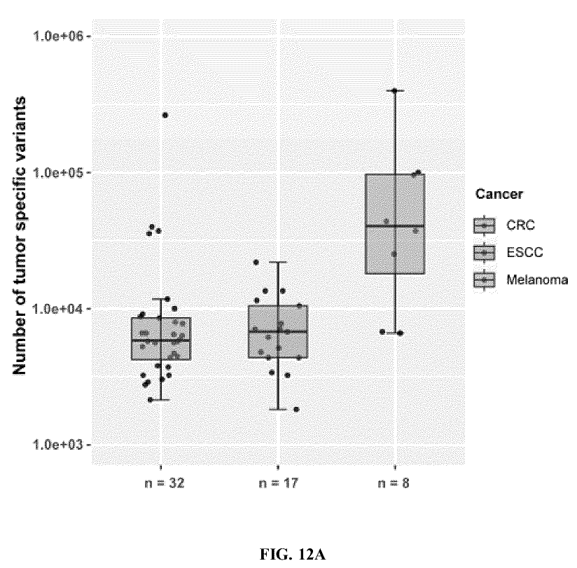

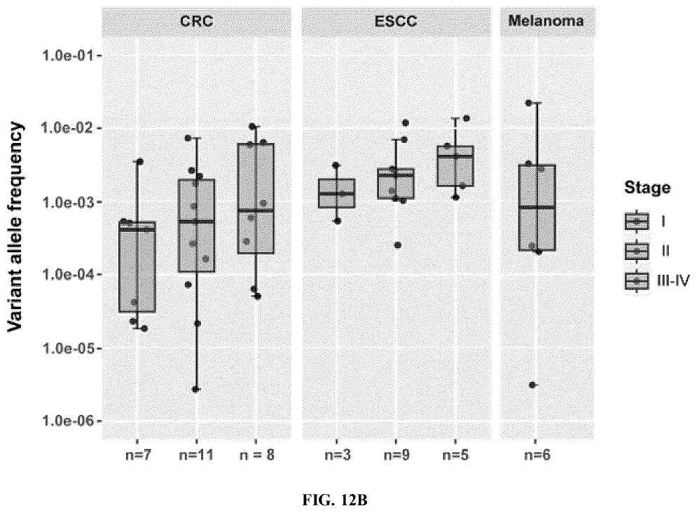

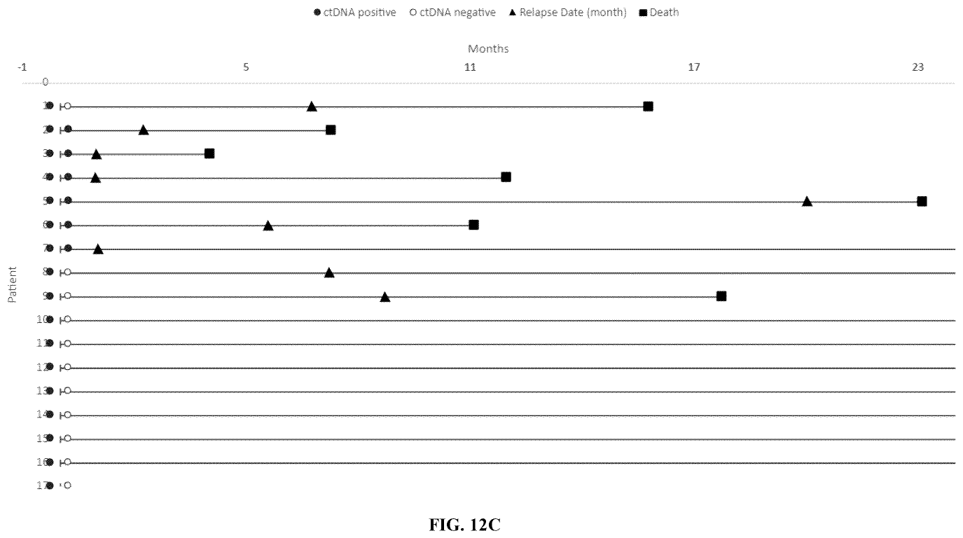

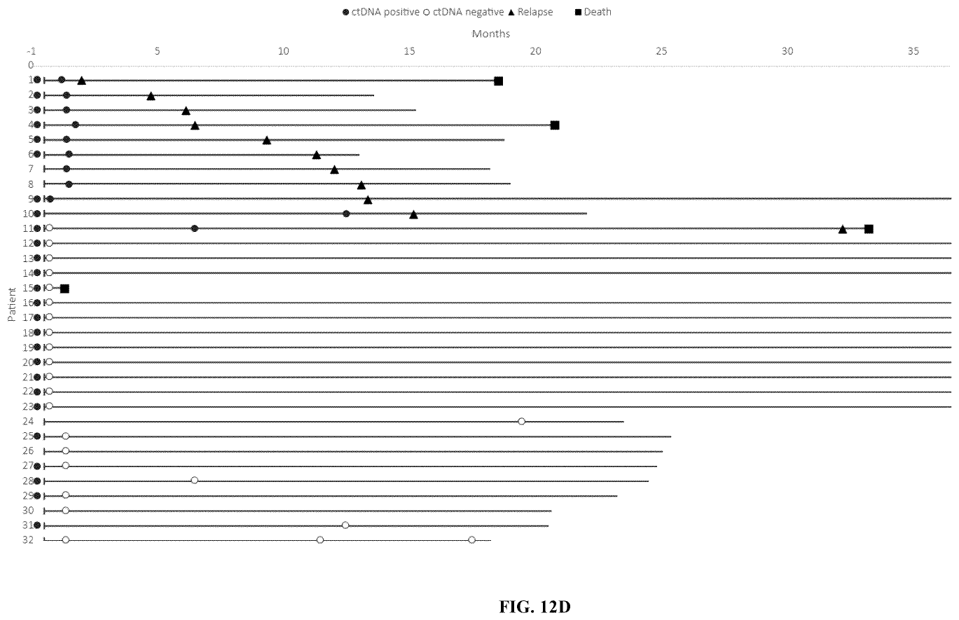

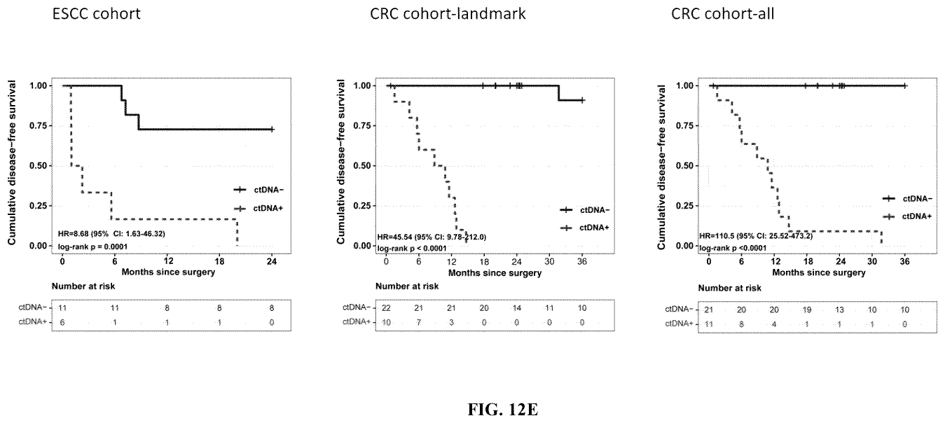

The ESCC cohort included patients from stage I-III (18% stage I, 53% stage II, 29% stage III) and received curative-intent surgery. Formalin Fixed Paraffin Embedded (FFPE), WBC, pre-Op plasma, and early (1-week) post-Op plasma samples were collected from all patients. Using tumor and WBC samples, we identified a median of 6768 tumor-specific variants per patient (FIG. 12A).

ctDNA was detected in all 17 of pre-Op samples with a median cTAF of 0.27% [Interquartile range (IQR): 0.13%-0.55%], with a non-significant trend to higher cTAF in later stage patients (FIG. 12B). In the post-surgery plasma samples, ctDNA was detected in 35.29% (6/17) of the patients, with a median cTAF of 1.3×10⁻⁴ (IQR: 1.9*10⁻⁵-1.1*10⁻²). The follow-up time of this ESCC cohort ranged from 4.03 months to 24 months. All the patients with ctDNA positive (ctDNA+) post-op samples (6/6, 100%) had a disease recurrence within 2 years of surgery; 5 of 6 (83%) patients had a recurrence within one year. (FIG. 12C). In contrast, of the 11 patients with ctDNA negative post-OP sample, only 3 had recurrence disease; all 8 disease-free patients were followed for 24 months. ctDNA detection at 1-week post-Op had 66.67% (95% CI: 29.93%-92.51%) sensitivity, 100% (95% CI: 63.06%-100%) specificity and 82.35% (95% CI: 56.57%-96.27%) accuracy in predicting ESCC recurrence.

The CRC patients were at diverse clinical stages (22% stage I, 38% stage II, 34% stage III, 6% stage IV) and received radical surgery. Formalin Fixed Paraffin Embedded (FFPE) samples were available from all patients. For the 15 patients with available WBC, the tumor-WBC workflow was used to identify tumor-specific variants; for all other patients, the first post-OP plasma samples were used for the WBC-free workflow (FIG. 11B). A median of 5820 tumor-specific variants per patient (2148-265800, FIG. 12A) were found, which correspond to ˜2 mutations/Mb.

Of the 32 patients, 26 had plasma samples collected before surgery and 28 had plasma samples collected at landmark (within one month of surgery). ctDNA was detected in all the pre-Op cfDNA samples with a median cTAF of 5.2×10⁻⁴ (IQR: 6.6×10⁻⁵-2.4×10⁻³), with a non-significant trend to higher cTAF in later stage patients (FIG. 12B). The median follow-up time in this CRC cohort was 24.13 months (IQR: 18.5-36). 34.4% (11/32) of patients had ctDNA detected in the post-Op samples. All patients that are ctDNA positive in the post-Op samples relapsed within 3 years after surgery (FIG. 12D). The median disease-free survival (DFS) of the ctDNA+ patient group was 10.8 months (IQR: 5.8-12.7), with 63.64% (7/11) of ctDNA+ patients had a recurrence within one year, and 90.91% (10/11) of ctDNA+ patients relapsed within two years. One ctDNA+ patient, patient #11, was ctDNA− at the first landmark timepoint, converted to ctDNA+ at 6 months post-Op and then relapsed at 32 months. Patients that were ctDNA negative (ctDNA−) at all post-Op time points were progression free during the follow up period (up to 36 months) (FIG. 12D). Taken together, these results suggest 90% (95% CI: 55.5%-99.8%) sensitivity at landmark, 100% sensitivity with longitudinal monitoring, 100% (95% CI: 80.5%-100%) specificity, and 96.3% (95% CI: 81%-99.9%) accuracy for predicting CRC recurrence.

When looking at early post-OP samples, MRD+ patients had shorter DFS times than MRD-patients in ESCC (hazard ratio, HR, 8.68, 95% CI: 1.63-46.32, log-rank p=0.0001) and CRC (HR, 45.54, 95% CI: 9.78-212, log-rank p<0.0001) (FIG. 12E).

ctDNA Monitoring During Immunotherapy

Advances in immune checkpoint blockade (ICB) have significantly improved survival of patients with advanced melanoma. However, only a fraction of patients (<20%) respond to ICB. There is an urgent need for means of prognosis and monitoring of patients undergo immunotherapy. Therefore, the use of AccuScan was explored for monitoring patient response to ICB in a pilot study with advanced melanoma (N=8). Total 22 plasma samples were collected, including 6 pre-treatment and 16 during treatment. WGS of the paired tumor and WBC DNA samples identified a median of 34323 SNVs, with an average of 90006 tumor-specific SNV per patient (FIG. 12A). All 6 pre-treatment samples were ctDNA positive with cTAF levels as low as 3.06×10⁻⁶ (FIG. 12B). Of the 16 samples taken during treatment, 10 were ctDNA positive.

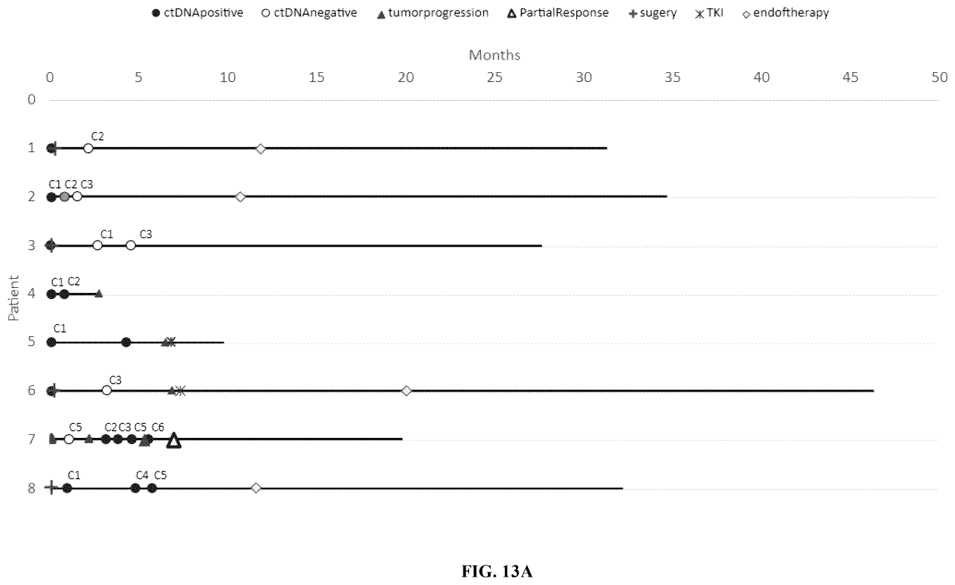

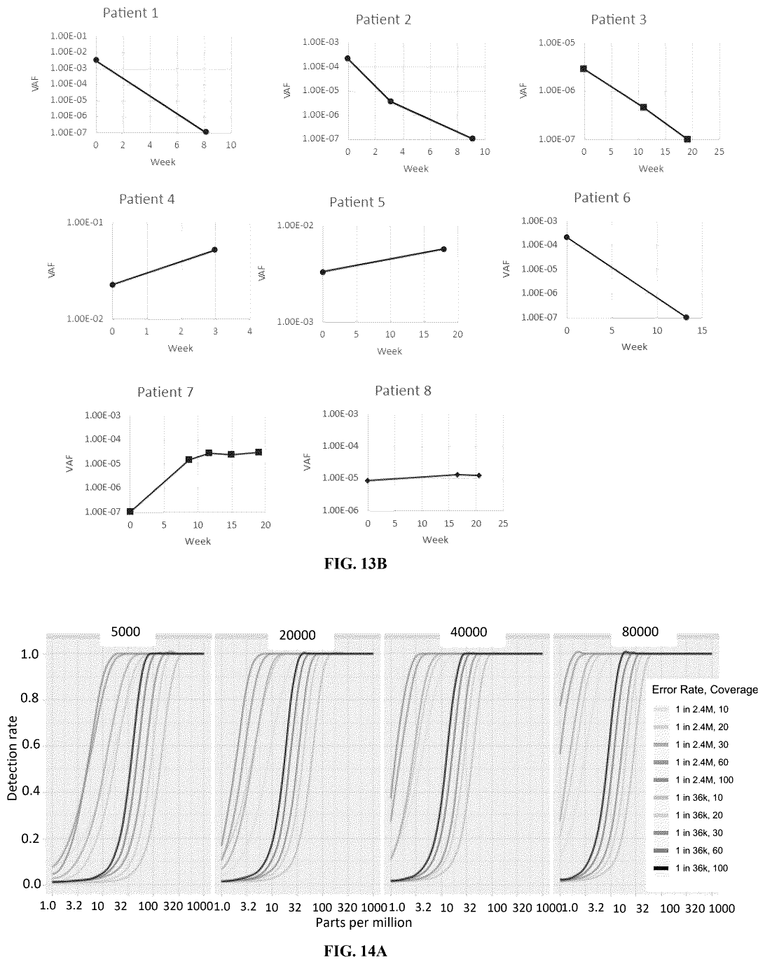

For patients 1 through 5 and 8, radiographic changes matched the AccuScan measured cTAF changes (FIGS. 13A-13B). Patients 1, 2, and 3 were ctDNA positive at pre-treatment timepoint, converted to ctDNA negative after treatment and had sustained complete response or no disease recurrence through the monitoring period. Patient 8 was ctDNA positive with persistently low cTAF (˜1×10⁻⁵) in samples taken during treatment (no pre-treatment sample was available) and CT scan showed stable lung nodules with no evidence of disease recurrence. Patients 4 and 5 had very high cTAF levels (0.4-16%) in all plasma samples and the cTAF of the second time point is about 2-fold or higher of the first time point. Both patients experienced tumor progression.

The correlations between ctDNA dynamics and radiographic information are complex for patients 6 and 7. In patient 6, AccuScan detected ctDNA at high cTAF (2×10⁴) before treatment and showed clearance of ctDNA 3 months after surgery and 2 cycles of ICI treatment (before cycle 3 of ICI treatment) (FIG. 13B), but the CT scan detected lymphadenopathy 4 months later (after 6 cycles of ICI treatment). The patient was switched to TKI and showed complete response (FIG. 13A). Despite the discordance between ctDNA and imaging data during treatment, the observed clearance of ctDNA is consistent with the clinical outcome. Either the MRD level failed to reflect tumor burden or the clinical response via imaging was delayed, and the patient was responding even before the switch to TKI.

Patient 7 did not have a pre-treatment sample, but the first sample during treatment was ctDNA negative, followed by 4 stably low-level ctDNA positive samples (FIG. 13B). Yet 3 CTs taken during treatment showed continuous tumor progression, although the fourth one taken after the last ctDNA test showed excellent partial response, and patient reached near CR 1 year later (FIG. 13A). This is another example showing discordance between imaging and ctDNA test, with ctDNA level remained steadily low while imaging showing tumor progression. It is possible that the ctDNA dynamic changes combined with imaging data may better predict patient outcome than either imaging or an isolated ctDNA result alone.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

1.-43. (canceled)

44. A method of detecting a tumor nucleic acid in a cell-free biological sample from a subject, the method comprising:
(a) sequencing nucleic acids derived from a tumor of said subject to obtain a tumor specific sequence variant;
(b) sequencing a nucleic acid derived from said cell-free biological sample, wherein said sequencing is at a depth of no greater than 18 reads per nucleic acid molecule;
(c) processing said sequence of said cell-free biological sample to identify said tumor specific sequence variant; and
(d) identifying said nucleic acid as having at least one said tumor specific sequence variant.

45. The method of claim 44, wherein said sequencing of (b) is at a depth of no greater than 5 reads per nucleic acid molecule.

46. The method of claim 44, further comprising calling said subject as minimum residual disease (MRD) positive when said nucleic acid derived from said cell-free biological sample has said at least one said tumor specific sequence variant.

47. The method of claim 44, wherein step (b) further comprises (i) circularizing said nucleic acid derived from said cell-free biological sample to create a circularized nucleic acid; (ii) amplifying said circularized nucleic acid to generate a concatemer comprising at least two copies of a sequence of said circularized nucleic acid, wherein sequencing said nucleic acid derived from said cell-free biological sample comprises sequencing said concatemer or a derivative thereof to obtain a sequence of said concatemer.

48. The method of claim 44, wherein the tumor specific sequence variant is identified by comparing a sequence from said nucleic acids derived from the tumor to a sequence from said nucleic acid derived from said cell-free biological sample.

49. The method of claim 44, wherein obtaining said tumor specific sequence variant comprises sequencing nucleic acids derived from a healthy tissue of said subject.

50. The method of claim 49, wherein said healthy tissue has low or no tumor content.

51. The method of claim 44, wherein said nucleic acids derived from said tumor are subjected to selection prior to sequencing in step (a).

52. The method of claim 51, wherein said selection comprises negative selection to remove non-target sequences from said nucleic acids.

53. The method of claim 51, wherein said selection comprises positive selection to select target sequences from said nucleic acids.

54. The method of claim 44, further comprising, prior to (b) subjecting said nucleic acid derived from said cell-free biological sample to selection.

55. The method of claim 54, wherein said selection comprises negative selection to remove non-target sequences from said nucleic acids.

56. The method of claim 55, wherein negative selection comprises annealing one or more blocking oligonucleotides to unwanted sequences in said nucleic acids derived from said tumor or said nucleic acids derived from said healthy tissue and circularizing remaining single stranded nucleic acids.

57. The method of claim 56, wherein said blocking oligonucleotides have modified 5′ ends, modified 3′ ends, or modified 5′ and 3′ ends.

58. The method of claim 54, wherein said selection comprises positive selection to select target sequences from said nucleic acids.

59. The method of claim 58, wherein said positive selection comprises amplifying said nucleic acids derived from said tumor with a plurality of random primers and a plurality of target specific primers.

60. The method of claim 44, wherein said cell-free biological sample is a bodily fluid.

61. The method of claim 60, wherein said bodily fluid comprises urine, saliva, blood, serum, or plasma.

62. The method of claim 44, wherein said tumor is a colorectal cancer, a pancreatic cancer, an ovarian cancer, a breast cancer, a prostate cancer, a bladder cancer, a lung cancer, a skin cancer, or a blood cancer.

63. A method of detecting a tumor nucleic acid in a cell-free biological sample from a subject, the method comprising:
(a) sequencing nucleic acids derived from a tumor of said subject to obtain a tumor specific sequence variant;
(b) sequencing a nucleic acid derived from said cell-free biological sample, wherein said sequencing is at a depth of no greater than 18 reads per nucleic acid molecule, wherein said sequencing is whole genome sequencing;
(c) processing said sequence of said cell-free biological sample to identify said tumor specific sequence variant; and
(d) identifying said nucleic acid as having at least one said tumor specific sequence variant.
